(19) United States
(12) Patent Application Publication
(10) Pub. No.: US 2019/0091320 A1
(43) Pub. Date: Mar. 28, 2019

(54) CHIMERIC PORCINE CIRCOVIRUS TYPE 2 (PCV2) VACCINES

(71) Applicant: VIRGINIA TECH INTELLECTUAL PROPERTIES, INC., BLACKSBURG, VA (US)

(72) Inventors: Xiang-Jin Meng, Blacksburg, VA (US); Shannon R. Matzinger, Denver, CO (US); Nathan M. Beach, Radford, VA (US)

(21) Appl. No.: 16/082,168

(22) PCT Filed: Mar. 3, 2017
(86) PCT No.: PCT/IB2017/000966

§ 371 Date: Sep. 4, 2018

Related U.S. Application Data
(60) Provisional application No. 62/304,596, filed on Mar. 7, 2016.

(51) Int. Cl. A61K 39/12; A61K 39/39; A61K 9/00

(52) U.S. Cl. CPC A61K 39/12; A61K 2039/552; A61K 9/0019; A61K 39/39

(57) ABSTRACT

Vaccine compositions and methods are described for providing immunity to porcine circovirus type two (PCV2) genotypes including by administration of a recombinant PCV2 capsid polypeptide which comprises antigenic epitopes from the capsids of multiple PCV2 genotypes. In other embodiments a recombinant chimeric porcine circovirus is provides for use as a vaccine that combines the nonpathogenic backbone of porcine circovirus type 1 (PCV1) with the sequences encoding a PCV2 capsid polypeptide comprises antigenic epitopes from the capsids of multiple PCV2 genotypes.

### CROSS REFERENCE TO RELATED APPLICATIONS

This application claims priority based on U.S. Provisional Application Ser. No. 62/304,596 filed Mar. 7, 2016, which is incorporated herein by reference it is entirety.

### REFERENCE TO SEQUENCE LISTING SUBMITTED ELECTRONICALLY

The Sequence Listing associated with the application is provided in text format in lieu of a paper copy, and is hereby incorporated by reference into the specification. The name of the text file containing the Sequence Listing is SequenceListing.txt. The text file is 42 kilobytes, was created on Feb. 24, 2017 and is being submitted electronically via EFS-Web.

### FIELD OF THE INVENTION

This invention relates generally to compositions and methods for prophylactic vaccines against mammalian circoviruses that cause considerable mortality and morbidity in swine farms.

### BACKGROUND OF THE INVENTION

Without limiting the scope of the invention, its background is described in connection with circovirus induced morbidity and mortality in swine populations. Porcine circovirus (PCV) is a small, non-enveloped, single-stranded DNA virus which belongs to the family Circoviridae. PCV type 1 (PCV1) was originally identified as a cell culture contaminant of the porcine kidney cell line PK-15 in the 1970's, and was later found to be non-pathogenic in pigs. In 1997, a pathogenic variant named PCV type 2 (PCV2) was identified in wasting piglets shortly after weaning. As more cases were identified worldwide, PCV2 was determined to be the primary causative agent of porcine circovirus-associated disease (PCVAD), which includes a broad spectrum of clinical symptoms such as wasting, reproductive failure, respiratory signs, enteritis, and the porcine dermatitis and nephropathy syndrome.

PCV2 is one of the most economically devastating viral pathogens to affect the global pig industry to date, and vaccination has been an effective strategy to reduce the economic losses associated with PCV2 infection. Currently, all commercially available inactivated or subunit vaccines target the PCV2a subtype. However, since 2005, a new subtype, PCV2b, has taken over as the most prevalent PCV2 strain associated with PCVAD cases in the U.S. and other countries. In addition, newly emerging PCV2d strains (previously referred to as “mutant PCV2b”), have been identified in an increasing number of cases in vaccinated herds worldwide, and some speculate may overcome vaccine protection. Until recently, only three PCV2 subtypes were recognized, including PCV2a, PCV2b, and PCV2c, the last of which was identified in Denmark during the 2000's but is not very prevalent. While the majority of the PCVAD cases in the United States are now associated with PCV2b, an emerging PCV2d subtype (previously referred to as “mutant PCV2b”) has been slowly increasing in the U.S. since its initial discovery in 2012 and is now more prevalent than PCV2a. See Xiao C T, Halbur P G, Opriessnig T. “Global molecular genetic analysis of porcine circovirus type 2 (PCV2) sequences confirms the presence of four main PCV2 genotypes and reveals a rapid increase of PCV2d” J. Gen. Virol. 96 (2015) 1830-1841. Although the exact reason for the emergence of PCV2d remains unclear, in can be commonly found in vaccinated herds, suggesting reduced protection against this emerging strain. In fact, a recent report has demonstrated the increasing genetic diversity amongst the PCV2d subtype, as the majority of isolates identified from 1999-2011 can be classified under the subclade “PCV2d-1,” and the majority of isolates identified recently, from 2006-2014, diverge from the PCV2d-1 subclade and are now designated “PCV2d-2”. See Xiao et al. 2015, supra. In addition, in vitro evidence suggests distinct antigenic differences among PCV2 subtypes, which may help explain the emergence of new strains. Therefore, in order to address the concern of emerging PCV2d as well as the predominant PCV2b now circulating in global swine herds, future vaccine strategies should focus on broadening the protection of a single vaccine by targeting emerging strains such as PCV2d and the predominant PCV2b subtype.

From the foregoing, it appeared to the present inventors that a new vaccine will be required to prevent outbreaks of porcine circovirus. Provided herein are such preventive vaccines.

### BRIEF SUMMARY OF THE INVENTION

The present invention provides a vaccine composition including a recombinant porcine circovirus type 2 (PCV2) capsid polypeptide that comprises antigenic epitopes from the capsids of multiple PCV2 genotypes.

In one embodiment, the present invention provides a vaccine composition comprising a recombinant porcine circovirus type 2 (PCV2) capsid polypeptide or an immunogenic derivative thereof, wherein the recombinant porcine circovirus type 2 (PCV2) capsid polypeptide is a chimeric amino acid sequence that differs from any naturally occurring PCV2 capsid polypeptide and includes capsid-derived amino acid sequences from multiple PCV2 genotypes.

In one embodiment, the vaccine composition comprises a recombinant PCV2 capsid polypeptide that is selected from capsid polypeptides designated as 3cl.14 (SEQ ID NO: 8), 3cl.13 (SEQ ID NO: 4), 3cl.4_2 (SEQ ID NO: 2), 3cl.12_2 (SEQ ID NO: 6).

In one embodiment, the recombinant PCV2 capsid polypeptide or immunogenic derivative thereof includes capsid-derived amino acid sequences from two or more of PCV2a, PCV2b, PCV2c, PCV2d and PCV2e parental genotypes.

In one embodiment, the recombinant PCV2 capsid polypeptide or immunogenic derivative thereof varies from any contributing parental genotype by 3-37 amino acids. In one embodiment, the immunogenic derivative varies from any parental chimeric capsid polypeptides by 1-27 amino acids.

In one embodiment, the recombinant PCV2 capsid polypeptide or immunogenic derivative thereof comprises capsid-derived amino acid sequences of at least PCV2c and PCV2d.

In one embodiment, the PCV2 capsid polypeptide is expressed in bacterial, yeast, mammalian, or insect cells.

In one embodiment, the recombinant PCV2 capsid polypeptide or immunogenic derivative thereof is encoded by a viral vector.

In one embodiment, the vaccine composition includes a recombinant chimeric porcine circovirus that combines a nonpathogenic backbone of porcine circovirus type 1 (PCV1) with a novel recombinantly generated porcine circovirus type 2 (PCV2) capsid polypeptide that comprises antigenic epitopes from the capsids of multiple PCV2 genotypes.

In one embodiment, the vaccine composition includes a recombinant chimeric porcine circovirus comprising a recombinant porcine circovirus type 1 (PCV1) that encodes a porcine circovirus type 2 (PCV2) capsid polypeptide in place of the capsid protein of PCV1, the PCV2 capsid polypeptide comprising epitopes from capsid polypeptides of multiple PCV2 genotypes. In one embodiment, the recombinant chimeric porcine circovirus is designated as PCV1_3cl.14 and is encoded by SEQ ID NO: 37.

In one embodiment, the PCV2 capsid polypeptide is encoded by a DNA-shuffled PCV2 capsid gene sequence.

In one embodiment, the PCV2 capsid polypeptide comprises antigenic epitopes from the capsids of at least two PCV2 genotypes selected from the group consisting of PCV2a, PCV2b, PCV2c, PCV2d and a recently identified divergent PCV2a virus previously referred to as “PCV2e.” As used herein, the terms “divergent PCV2a virus” and “PCV2e” are used interchangeably. In another embodiment, the PCV2 capsid polypeptide comprises antigenic epitopes from the capsids of at least PCV2c and PCV2d strains.

In one embodiment, the vaccine is a live vaccine, a modified live vaccine, an inactivated vaccine, an attenuated vaccine, or a subunit vaccine. In another embodiment, the vaccine composition comprises a viral vector encoding a PCV2 capsid polypeptide.

In one specific embodiment, the vaccine is an inactivated vaccine. In another specific embodiment, the vaccine is a subunit vaccine or an inactivated whole virus vaccine.

In one aspect, a vaccine composition according to the invention further includes an adjuvant. In one embodiment, the adjuvant is selected from: an oil-in-water adjuvant, a polymer and water adjuvant, a water-in-oil adjuvant, an aluminum hydroxide adjuvant, a vitamin E adjuvant and combinations thereof. In one specific embodiment, the adjuvant comprises an oil emulsion that includes a polyoxyethylene-polyoxypropylene block copolymer, squalane, polyoxyethylene sorbitan monooleate and a buffered salt solution (SP-oil). In one embodiment, the composition further includes a pharmaceutically acceptable carrier.

In another embodiment, the vaccine composition further includes at least one additional antigen. In certain embodiments, the at least one additional antigen is protective against a microorganism that can cause disease in pigs. In another embodiment, the antigen comprises one or more antigens derived from bacterial, viral, or parasitic microorganisms that are known to be pathogenic in pigs. For example, the bacterial microorganism may be selected from one or more of Actinobacilllus pleuropneumoniae, Bordetella bronchiseptica, Brachyspira hyodysenteriae, Brucellosis (B. suis), Campylobacter spp., Clostridium spp., Escherichia coli, Erysipelothrix rhusiopathiae, Haemophilus parasuis, Isospora suis, Lawsonia intracellularis, Leptospira spp., Listeria monocytogenes, Mycoplasma hyorhinis, Mycoplasma hyosynoviae, Mycoplasma flocculare, Mycoplasma hyopneumoniae, Pasteurella multocida, Streptococcum suis, Staphylococcus spp. including S. aureus and S. hyicus, Salmonella spp. including S. choleraesuis and S. enteritidis, methicillin-resistant Staphylococcus aureus (MRSA), Trichinella spiralis, Toxoplasma gondii and Yersinia enterocolitica. Viral microorganisms may include African swine fever virus, classical swine fever virus (CSF), foot and mouth disease virus (FMDV), Nipah virus, porcine cytomegalovirus, porcine epidemic diarrhea virus (PEDV), porcine enteroviruses, encephalomyocarditis virus, porcine reproductive and respiratory syndrome virus (PRRSV), porcine parvovirus (PPV), porcine respiratory coronavirus (PRVV), pseudorabies virus (PRV) a.k.a. suid herpesvirus type 1, rotavirus, swine influenza virus (SIV), torque teno virus (TTV), and transmissible gastroenteritis virus of swine (TGEV). Parasitic microorganisms may include Ascaris suum, Balatidium coli, Toxiplasma gondii, and Cryptosporidium parvum.

In further embodiments, chimeric nucleic acid molecules are provided having a DNA-shuffled PCV2 capsid gene sequence derived from multiple PCV2 genotypes or a derivative thereof that encodes epitopes of PCV2 capsid polypeptides. In one embodiment, these chimeric nucleic acid molecules comprise nucleic acid molecules encoding a nonpathogenic PCV1 that contains a DNA-shuffled PCV2 capsid gene sequence derived from multiple PCV2 genotypes in place of the capsid gene sequence of the PCV1 nucleic acid molecule. In one embodiment, the DNA-shuffled PCV2 capsid gene sequence is selected from capsid gene sequences designated as 3cl.14 (SEQ ID NO: 7), 3cl.13 (SEQ ID NO: 3), 3cl.4_2 (SEQ ID NO: 1), 3cl.12_2 (SEQ ID NO: 5).

In certain embodiments, the chimeric nucleic acid molecules are included in a viral vector. Examples include but are not limited to baculovirus vectors and parapox vectors.

Also provided herein are methods of protecting pigs against infection by multiple PCV2 genotypes. Methods include administering to pigs an immunologically effective amount of a vaccine composition, a chimeric nucleic acid molecule, or viral vector disclosed herein. In various embodiments, the methods include administering the vaccine composition, chimeric nucleic molecule or viral vector to the pig by one or more routes selected from parenterally, intranasally, intradermally and transdermally. In another embodiment, the vaccine composition, chimeric nucleic molecule or viral vector is administered in a single dose. In another embodiment, the composition is administered in conjunction with at least one additional antigen that is protective against a microorganism that can cause disease in pigs, examples of which are described above. In yet another embodiment, the pig is protected against infection by at least PCV2b and PCV2d.

### BRIEF DESCRIPTION OF THE DRAWINGS

For a more complete understanding of the present invention, including features and advantages, reference is now made to the detailed description of the invention along with the accompanying figures:

FIG. 1 shows the results of seroconversion to PCV2-specific antibodies in pigs experimentally infected with chimeric PCV2 viruses containing shuffled capsids or with the PCV1-2a vaccine virus.

FIG. 2 shows the results of seroconversion to PCV2-specific antibodies by ELISA and detection of viremia by PCR in pigs vaccinated with PCV1-3cl14 virus and challenged with PCV2b or PCV2d-2.

FIG. 3 depicts an amino acid sequence alignment of the capsid proteins from the five parental PCV2 wild-type strains and the four novel DNA shuffled capsid proteins disclosed herein. The first five sequences represent the parental strains while the bottom four sequences represent the DNA-shuffled PCV2 capsids. Amino acids that differ from the consensus are shown in black.

FIG. 4 depicts a phylogenetic tree of the capsid genes of selected PCV2 strains from different subtypes. The phylogenetic tree was constructed using the neighbor-joining method with bootstraps in 1,000 replicates. The number above each major branch indicates the bootstrap value. The bold italicized sequence names represent the PCV2 sequences of the five parental strains used for DNA shuffling as disclosed herein.

FIG. 5A-FIG. 5D compare the 50% neutralizing antibody titers against four PCV2 wild-type strains from sera of pigs experimentally inoculated with chimeric viruses PCV2-3cl13, PCV2-3cl14, PCV2-3cl4_2, and PCV2-3cl12_2, or PCV1-2a with shuffled capsid genes. In vitro 50% neutralization assay of respective sera collected at 56 days post-infection against three parental PCV2 strains: (FIG. 5A) PCV2a, (FIG. 5B) PCV2d-1, (FIG. 5C) PCV2b, and (FIG. 5D) PCV2d-2 isolate. The NA titers were calculated as the highest 2-fold dilution (2ⁿ) of the serum sample that showed a 50% or greater reduction in the number of positive fluorescent foci, compared to the serum samples from the mock (PBS) inoculated control group in the same dilution. The asterisk (*) indicates p<0.05 analyzed using one-way ANOVA.

FIG. 6 depicts the PCV2 capsid-specific antibody response in conventional pigs experimentally inoculated with the chimeric virus PCV1-3cl14 vaccine candidate and challenged with wild-type virus strains PCV2b or PCV2d-2. The mean S/P ratio±SEM is plotted for each treatment group throughout the duration of the study. The virus challenge took place at 42 days post-vaccination (dpv). The dashed line at 0.2 S/P ratio denotes the lower end cutoff for a positive sample in this assay.

FIG. 7A shows quantification using qPCR of PCV2 viral DNA loads in sera from pigs vaccinated with the chimeric PCV1-3cl14 virus and subsequently challenged with PCV2b compared to challenge only controls. FIG. 7B shows quantification using qPCR of PCV2 viral DNA loads in sera from pigs vaccinated with the chimeric PCV1-3cl14 virus and subsequently challenged with PCV2d-2 compared to challenge only controls. In FIG. 7A and FIG. 7B, group means±SEM are plotted for each time point post-challenge. The limit of detection for the assay was 10^{4.2} copies/mL serum of ORF1 DNA determined by a standard curve for 10¹-10¹⁰ copies of the wild-type PCV2b genome. (*) indicates statistical significance between groups (Student's t-test, corrected for multiple tests).

FIG. 8A shows quantification using qPCR of PCV2 viral DNA loads in lymph nodes from pigs vaccinated with the chimeric PCV1-3cl14 virus and challenged with PCV2b compared to challenge only controls. FIG. 8B shows quantification using qPCR of PCV2 viral DNA loads in lymph nodes from pigs vaccinated with the chimeric PCV1-3cl14 virus and challenged with PCV2d-2 compared to challenge only controls. In FIG. 8A and FIG. 8B, group means±SEM are plotted for each time point post-challenge. The limit of detection for the assay was 10^{7.1} copies/mg tissue of ORF1 viral DNA, as determined by a standard curve for 10¹-10¹⁰ copies of the wild-type PCV2b genome. The asterisk (*) indicates statistical significance between groups at that time point (Student's t-test, corrected for multiple tests).

FIG. 9A-FIG. 9F compare lymphoid tissues in pigs vaccinated with the chimeric PCV1-3cl.14 virus and subsequently challenged with PCV2b or PCV2d-2 with those of challenge only controls. FIG. 9A shows lymphoid depletion in lymph nodes. FIG. 9B shows histiocytic replacement in lymph nodes. FIG. 9C shows lymphoid depletion in the spleen. FIG. 9D shows histiocytic replacement in the spleen. FIG. 9E shows lymphoid depletion in tonsils. FIG. 9F shows histiocytic replacement in tonsils. In each of FIG. 9A-FIG. 9F, necropsy results were compared for vaccinated and challenged animals (▪) with those of challenge only controls (∘). Individual animal scores are represented by individual symbols and group means±SEM are displayed. The asterisk (*) indicates statistically significant differences between groups (student's t-test).

FIG. 10A-FIG. 10C depict quantification of PCV2 viral antigen in lymphoid tissues by PCV2 immunohistochemistry (IHC). The tissues were obtained from pigs vaccinated with the chimeric PCV1-3cl14 virus and subsequently challenged with PCV2b or PCV2d-2 compared to challenge only controls. FIG. 10A shows PCV2 viral antigen scores determined for lymph nodes, FIG. 10B shows PCV2 viral antigen scores determined the spleen, and FIG. 10C shows PCV2 viral antigen scores determined for tonsils. In each of FIG. 10A-FIG. 10C, necropsy results were compared for vaccinated and challenged animals (▪) with those of challenge only controls (∘). Individual animal scores are represented by individual symbols and group means±SEM are displayed. The asterisk (*) indicates statistically significant differences between groups (student's t-test).

FIG. 11 shows the nucleic acid (SEQ ID NO: 1), polypeptide sequence (SEQ ID NO: 2) and the aligned nucleic acid/amino acid translation for the capsid protein of the chimeric PCV2-3cl.4_2 virus, while FIG. 12 shows the full length nucleic acid sequence of the PCV2-3cl.4_2 virus (SEQ ID NO: 33).

FIG. 13 shows the nucleic acid (SEQ ID NO: 3), polypeptide sequence (SEQ ID NO: 4) and the aligned nucleic acid/amino acid translation for the capsid protein of the chimeric PCV2-3cl.13 virus, while FIG. 14 shows the full length nucleic acid sequence of the PCV2-3cl.13 virus (SEQ ID NO: 34).

FIG. 15 shows the nucleic acid (SEQ ID NO: 5), polypeptide sequence (SEQ ID NO:6) and the aligned nucleic acid/amino acid translation for the capsid protein of the chimeric PCV2-3cl.12_2 virus, while FIG. 16 shows the full length nucleic acid sequence of the PCV2-3cl.12_2 virus (SEQ ID NO: 35).

FIG. 17 shows the nucleic acid (SEQ ID NO: 7), polypeptide sequence (SEQ ID NO: 8) and the aligned nucleic acid/amino acid translation for the capsid protein of the chimeric PCV2-3cl.14 virus, while FIG. 18 shows the full length nucleic acid sequence of the PCV2-3cl.14 virus (SEQ ID NO: 36).

FIG. 19 shows the full length nucleic acid sequence of the PCV1-3cl.14 virus (SEQ ID NO: 37), where the capsid sequence of the PCV2-3cl.14 virus is cloned onto a PCV1 backbone.

FIG. 20A-FIG. 20C shows an alignment between PCV1_3cl.14 full length as the Query sequence with PCV2_3cl.14 full length as the Subject sequence. The sequence of the capsid protein of the chimeric PCV2-3cl14 is underlined and bold.

FIG. 21 shows the aligned nucleic acid (SEQ ID NO: 9)/amino acid translation (SEQ ID NO: 10) for the capsid protein of the PCV2a genotype virus reflected in GenBank AF264042.

FIG. 22 shows the aligned nucleic acid (SEQ ID NO: 11)/amino acid translation (SEQ ID NO: 12) for the capsid protein of the PCV2b genotype virus reflected in GenBank GU799576.

FIG. 23 shows the aligned nucleic acid (SEQ ID NO: 13)/amino acid translation (SEQ ID NO: 14) for the capsid protein of the PCV2d-1 genotype virus reflected in GenBank AY181947.

FIG. 24 shows the aligned nucleic acid (SEQ ID NO: 15)/amino acid translation (SEQ ID NO: 16) for the capsid protein of the PCV2c genotype virus reflected in GenBank EU148503.

FIG. 25 shows the aligned nucleic acid (SEQ ID NO: 17)/amino acid translation (SEQ ID NO: 18) for the capsid protein of the PCV2e genotype virus reflected in GenBank EU524533.

### DETAILED DESCRIPTION OF THE INVENTION

While the making and using of various embodiments of the present invention are discussed in detail below, it should be appreciated that the present invention provides many applicable inventive concepts which can be employed in a wide variety of specific contexts. The specific embodiment discussed herein are merely illustrative of specific ways to make and use the invention and do not delimit the scope of the invention.

Provided herein are disclosures of molecularly breeding genetically-divergent PCV2 strains representing PCV2a, PCV2b, PCV2c, PCV2d and “PCV2e” subtypes by DNA shuffling of the capsid genes to produce novel chimeric PCV2 capsid sequences representing the genetic diversity among PCV2 subtypes. It is believed that this is the first disclosure of construction of an effective chimeric PCV2 vaccine candidate by shuffling the capsid genes of five (5) divergent PCV2 strains belonging to different subtypes.

In one embodiment, when placed in a PCV2a backbone, a chimeric virus (PCV2-3cl14) induced high neutralizing antibody titers against different PCV2 subtypes. In another embodiment, a candidate vaccine (PCV1-3cl14) was produced by cloning the shuffled 3cl14 capsid into the backbone of a non-pathogenic PCV1. A vaccine efficacy study revealed that chimeric virus PCV1-3cl14 induced protective immunity against challenge with PCV2b or PCV2d in pigs thus representing a strong candidate for a novel vaccine in pigs infected with variable PCV2 strains.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used in the specification and claims, the singular form “a”, “an” and “the” include plural references unless the context clearly dictates otherwise. For example, the term “a protein antigen” includes a plurality of protein antigens, including mixtures thereof.

As used herein, the term “comprising” is intended to mean that the compositions and methods include the recited elements, but do not exclude other elements.

The term “antigen” refers to a compound, composition, or immunogenic substance that can stimulate the production of antibodies or a T-cell response, or both, in an animal, including compositions that are injected or absorbed into an animal. The immune response may be generated to the whole molecule, or to a portion of the molecule (e.g., an epitope or hapten).

As used herein the terms “epitope” refers to discrete portions of antigens that serve as binding sites recognized by antibodies or T-cell receptors. For example, an epitope can refer to the antigenic determinant recognized by the Complimentary Determining Regions (CDRs) of an antibody. The epitope binding aspect of the respective binding antibody is termed a “paratope.”

B-cell epitopes can be linear or conformational with the majority of strong epitopes being conformational. T-cell epitopes are linear. Linear epitopes are a continuous sequence of amino acids typically of 5-20 amino acids in length situated in a larger protein. Typically strong B-cell epitopes are defined by a conformational structure on the native protein. The amino acids forming the conformational epitope can be a folded continuous stretch of amino acids or may be discontinuous where the paratope recognizing aspects are located on disparate parts of the protein that are brought together by the folding of the native protein structure.

Due to unique tertiary conformational structures formed by the assembly of capsid proteins into intact assembled virion particles, viral epitopes can include cryptotopes, which are epitopes hidden in the assembled virion, and neotopes, which are epitopes formed only in the intact assembled virion but absent in its disassembled components. The term epitope as used herein includes cryptotopes and neotopes. For one non-limiting example, an “epitope” as used herein refers to a region of the PCV2 ORF2 shuffled capsid with which a paratope of a PCV2 capsid specific antibody reacts.

As used herein, the term “mimotope” refers to a macromolecule, most often a peptide, which mimics the conformational structure of an epitope and is bound by an antibody that recognizes a corresponding epitope. Thus mimotopes can be obtained by biopanning phage or phagemid display libraries using an antibody specific to a given epitope.

As defined herein, an “immunogenic or immunological composition”, refers to a composition of matter that comprises at least one antigen which elicits an immunological response in the host of a cellular and or antibody-mediated immune response to the composition or vaccine of interest.

The term “immune response” as used herein refers to a response elicited in an animal. An immune response may refer to cellular immunity (CMI) or humoral immunity or may involve both. The present invention also contemplates a response limited to a part of the immune system. Usually, an “immunological response” includes, but is not limited to, one or more of the following events: the production or activation of antibodies, B cells, helper T cells, suppressor T cells, and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein the term “antibody” includes both intact immunoglobulin molecules as well as portions, fragments, and derivatives thereof, such as, for example, Fab, Fab′, F(ab′)₂, Fv, Fsc, CDR regions, or any portion of an antibody that is capable of binding an antigen or epitope including chimeric antibodies that are bi-specific or that combine an antigen binding domain originating with an antibody with another type of polypeptide. The term “antibody” as used herein also includes single-domain antibodies (sdAb) and fragments thereof that have a single monomeric variable antibody domain (V_{H}) of a heavy-chain antibody. sdAb, which lack variable light (V_{L}) and constant light (C_{L}) chain domains are natively found in camelids (V_{H}H) and cartilaginous fish (V_{NAR}) and are sometimes referred to as “Nanobodies” by the pharmaceutical company Ablynx who originally developed specific antigen binding sdAb in llamas. The modifier “monoclonal” indicates the character of the antibody as being obtained from a substantially homogeneous population of antibodies originally produced from a single B-lymphocyte clone, and is not to be construed as requiring production of the antibody by any particular method. The term “monoclonal antibody” includes chimeric antibodies derived from monoclonal antibodies and species adapted monoclonal antibodies, as well as fragments, portions, regions, or derivatives thereof, provided by any known technique including but not limited to molecular techniques including phage display, affinity maturation and directed mutagenesis and chemical techniques including covalent modification and conjugation.

An “adjuvant” as used herein means a composition comprised of one or more substances that enhances the immune response to an antigen(s). The mechanism of how an adjuvant operates is not entirely known. Some adjuvants are believed to enhance the immune response by slowly releasing the antigen, while other adjuvants are strongly immunogenic in their own right and are believed to function synergistically.

As used herein, the term “multivalent” means a vaccine containing more than one antigen whether from the same species (i.e., different isolates of Mycoplasma hyopneumoniae), from a different species (i.e., isolates from both Pasteurella hemolytica and Pasteurella multocida), or a vaccine containing a combination of antigens from different genera (for example, a vaccine comprising antigens from Pasteurella multocida, Salmonella spp, Escherichia coli, Haemophilus somnus and Clostridium spp.).

The term “pig” or “piglet” as used herein means an animal of porcine origin.

As used herein, the term “virulent” means an isolate that retains its ability to be infectious in an animal host.

“Inactivated vaccine” means a vaccine composition containing an infectious organism or pathogen that is no longer capable of replication or growth. The pathogen may be bacterial, viral, protozoal or fungal in origin. Inactivation may be accomplished by a variety of methods including freeze-thawing, chemical treatment (for example, treatment with thimerosal or formalin), sonication, radiation, heat or any other convention means sufficient to prevent replication or growth of the organism while maintaining its immunogenicity.

The term “variant” as used herein refers to a polypeptide or a nucleic acid sequence encoding a polypeptide that has one or more amino acid variations, such as conservative variations or other minor modifications (e.g., truncations) such that the corresponding polypeptide has substantially equivalent function when compared to the wild-type polypeptide.

“Conservative variation” denotes the replacement of an amino acid residue by another biologically similar residue, or the replacement of a nucleotide in a nucleic acid sequence such that the encoded amino acid residue does not change or is another biologically similar residue. Examples of conservative variations include the substitution of one hydrophobic residue, such as isoleucine, valine, leucine or methionine for another hydrophobic residue, or the substitution of one polar residue, such as the substitution of arginine for lysine, glutamic acid for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

As used herein the term “shuffled” means molecular recombination between fragments of capsid genes of different PCV strains. Shuffling can be conducted by several presently known methods including: PCR shuffling, a staggered extension process (StEP), synthetic shuffling, incremental truncation for the creation of hybrid enzymes (ITCHY), and non-homologous random recombination (NRR). Regardless of how the shuffling is undertaken, the resulting molecule is termed a “chimera” because it is composed of recombined pieces of different parental DNAs. PCR shuffling is conducted by fragmentation such as by digestion with DNase, mixing of the fragments and amplification by primer-less PCR followed by amplification using primers specific to regions on the ends of the gene to be recombined by shuffling. The final primers will include restriction enzyme site that enable cloning of the shuffled and resembled fragments. This method was first described by Stemmer, W. P. C. “DNA shuffling by random fragmentation and reassembly: In vitro recombination for molecular evolution” Proc. Natl. Acad. Sci. USA 91 (22) (1994) 10747-10,751. The StEP method obtains homologous recombination by repeated premature denaturation PCR extension stages. The partially polymerized genes can switch templates and the obtained full-length chimeras have varying numbers of crossovers. Synthetic shuffling, also known as assembly PCR, utilizes overlapping libraries of oligonucleotides that encode genetic variations at a number of loci. Synthetic shuffling can yield considerable variation. ITCHY involves use of exonucleases to truncate mixtures of different gene variants followed by blunt-end ligation. NRR uses DNase fragmentation followed by blunt-end ligation to generate diverse topological rearrangements (deletions, insertions and domain reordering).

As used herein, the terms “pharmaceutically acceptable carrier” and “pharmaceutically acceptable vehicle” are interchangeable and refer to a fluid vehicle for containing vaccine antigens that can be injected into a host without adverse effects. Suitable pharmaceutically acceptable carriers known in the art include, but are not limited to, sterile water, saline, glucose, dextrose, or buffered solutions. Carriers may include auxiliary agents including, but not limited to, diluents, stabilizers sugars and amino acids), preservatives, wetting agents, emulsifying agents, pH buffering agents, viscosity enhancing additives, colors and the like.

As used herein, the term “vaccine composition” includes at least one antigen or immunogen in a pharmaceutically acceptable vehicle useful for inducing an immune response in a host. Vaccine compositions can be administered in dosages and by techniques well known to those skilled in the medical or veterinary arts, taking into consideration such factors as the age, sex, weight, species and condition of the recipient animal, and the route of administration. The route of administration can be percutaneous (through the skin via intradermal, transdermal, subcutaneous, intramuscular routes or mucosal via oral, nasal, anal, vaginal) or via a parenteral route (intravenous or intraperitoneal). Vaccine compositions can be administered alone, or can be co-administered or sequentially administered with other treatments or therapies. Forms of administration may include suspensions, syrups or elixirs, and preparations for parenteral, subcutaneous, intradermal, intramuscular or intravenous administration (e.g., injectable administration) such as sterile suspensions or emulsions. Vaccine compositions may be administered as a spray or mixed in food and/or water or delivered in admixture with a suitable carrier, diluent, or excipient such as sterile water, physiological saline, glucose, or the like. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard pharmaceutical texts, such as “Remington's Pharmaceutical Sciences,” 1990 may be consulted to prepare suitable preparations, without undue experimentation.

Porcine circovirus type 2 (PCV2) is the primary causative agent of porcine circovirus-associated disease (PCVAD) causing high economic losses in the global swine industry when uncontrolled. Indeed, PCVAD is arguably one of the most economically-important diseases affecting the global swine industry. Characterized by progressive wasting, hallmark histological lesions of lymphoid depletion with histiocytic infiltration, and the presence of PCV2 antigen or DNA in the lesions, PCVAD is caused by PCV2 infection, although co-infection with other pathogens are usually necessary for the development of the full-spectrum of clinical PCVAD. Currently available commercial vaccines all target the PCV2a subtype, which prior to 2005 was the main subtype, even though the predominant subtype currently circulating in the global pig population is PCV2b, and the emerging PCV2d subtype is increasingly associated with PCVAD in vaccinated herds. Now PCV2b has surpassed PCV2a as the most prevalent strain associated with PCVAD losses in the swine industry. In addition, recently, evidence of vaccine failures has been reported, and has been associated with the emergence of the PCV2d (or mutant PCV2b) subtype. Therefore, it is imperative to develop the next generation of vaccines especially against the emerging PCV2 strains.

To this end, the present inventors undertook to molecularly breed the capsid genes from different PCV2 subtypes by DNA shuffling, and to develop a candidate chimeric virus vaccine based on the non-pathogenic PCV1 backbone and shuffled capsid genes of divergent PCV2 subtypes. In the DNA shuffling approach, five genetically distinct capsid sequences from each of the four known PCV2 subtypes, as well as from the “PCV2e” type, which is now generally considered as a divergent PCV2a strain, were used. Of the more than fifty (50) shuffled PCV2 capsids that were cloned and sequenced, infectious chimeric viruses were rescued in PK15 cells only in four of them, suggesting that the small PCV2 genome cannot support a large number of forced random reassortments within the capsid gene.

The four viable chimeric viruses with shuffled PCV2 capsids generated by DNA shuffling contained antigenic epitopes from all five genetically divergent PCV2 strains, although most of the variability in the shuffled capsids could be found in the PCV2c parental strain. PCV2c subtype is the most divergent strain from the rest of the PCV2 subtypes identified thus far, based on a phylogenetic analysis. Alignment of the five selected parental strains revealed that the PCV2c does, in fact, contain the most genetically distinct amino acid variations, though some of these amino acids overlap with the parental PCV2d strain, including the addition of a terminal lysine residue. The presence of amino acid residues unique to PCV2c and PCV2d strains suggests that, although the PCV2c subtype has not associated with any clinical disease, this subtype could have contributed to the evolutionary emergence of the current PCV2d subtype. In fact, the PCV2c subtype was recently isolated from feral pigs in Brazil for first time since it was originally described in Denmark in the early 1990s. The feral pig populations were also infected with the other three PCV2 subtypes, suggesting the possibility of recombination. Therefore, these findings supported the inclusion of PCV2c for DNA shuffling in order to increase the breadth of protection of the resulting candidate vaccine against currently emerging and future possible emerging PCV2 strains.

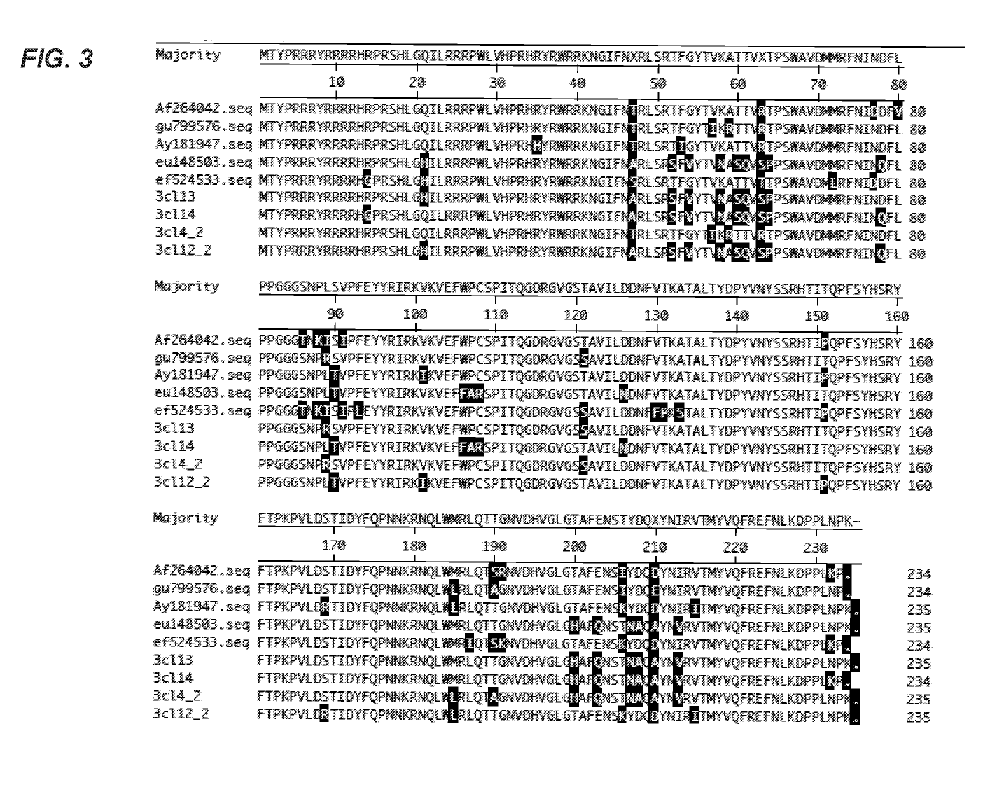

In order to determine the in vivo infectivity of the shuffled chimeric viruses and to screen for the best chimera for subsequent challenge and efficacy study, conventional pigs were experimentally inoculated in a pilot study with each of the four chimeric viruses with shuffled capsids in the PCV2a backbone as well as with a chimeric PCV1-2a vaccine virus. The results showed that chimeric virus PCV2-3cl.14 induced higher levels of neutralizing antibody titers when compared to the chimeric PCV1-2a virus, as well as the other three chimeric viruses. The chimeric virus PCV2-3cl.14 also induced significantly higher neutralizing antibody titers against PCV2a and PCV2d-2 strains. Comparison of the amino acid sequences of the shuffled capsid 3cl.14 to the other three shuffled capsids as well as the PCV1-2a reveals three regions with distinct amino acid residues as shown on FIG. 3. Two of these regions, amino acids 106-108 and 126, overlap with previously-identified B-cell antigenic epitopes. In addition, a mutation at position 126 corresponded to a location within the predicted B-cell and SLA-class II epitopes. Interestingly, the 3cl.13, 3cl.4_2, and 3cl.12_2 shuffled capsids all contain the additional lysine residue at the C-terminus of the capsid found in the PCV2d parental strain. This mutation is suggested to play a role in the increased pathogenicity and vaccine failure of the emerging PCV2d strains, although no direct evidence of this role has been reported to date. However, the 3cl.14 shuffled capsid sequence does not include the additional lysine, suggesting that it is not a necessary epitope for producing neutralizing antibodies against the PCV2d-2 strains, since PCV1-3cl.14 protects against PCV2d-2 infection in the challenge and efficacy experiment. It is possible that the properties of 3cl.14 capsid sequence discussed above are important for production of cross-protective neutralizing antibodies in pigs.

Based on induction of significantly higher cross-neutralizing antibody titers, the shuffled 3cl.14 capsid sequence was subsequently selected to produce a chimeric virus PCV1-3cl.14 vaccine candidate. The protective efficacy of the PCV1-3cl.14 chimeric virus as a potential vaccine was evaluated by challenging vaccinated pigs with PCV2b or PCV2d, respectively. PCV2b is the predominant subtype currently infecting pigs worldwide, whereas the PCV2d is an emerging subtype. We previously demonstrated the attenuation of chimeric PCV1-2a and PCV1-2b viruses in the genomic backbone of the non-pathogenic PCV1 in vivo. Consistent with these previous reports, there was no detectable PCV1-3cl.14 viremia in vaccinated pigs throughout the duration of the study, and no detectable clinical disease prior to challenge with either PCV2b or PCV2d (data not shown), even though the vaccinated pigs are infected as evidenced by seroconversion to PCV2 capsid antibody.

Vaccination with the chimeric virus PCV1-3cl.14 vaccine candidate resulted in significantly reduced PCV2b or PCV2d viral DNA loads at the peak of viremia as well as reduced viral DNA loads in lymphoid tissues at termination of the study. Furthermore, the lymphoid lesions were also significantly reduced in vaccinated groups subsequently challenged with PCV2b compared to mock-vaccinated and challenged controls. Though the vaccinated animals showed no significant reduction in spleen lymphoid depletion and spleen and tonsil histiocytic replacement when challenged with PCV2d, they did have significant reduction for the rest of the PCVAD scores, as well as reduced viral DNA loads in serum and lymph node tissues, indicating that the PCV1-3cl.14 chimeric virus vaccine candidate induced protection against both PCV2b and PCV2d challenge in conventional pigs.

In summary, this is the first disclosure to our knowledge of the construction of a viable chimeric PCV2 vaccine candidate by shuffling the capsid gene of 5 divergent PCV2 strains belonging to different subtypes. Importantly, vaccination of pigs with a chimeric virus PCV1-3cl.14 with shuffled capsid genes induced protective immunity against challenge with PCV2b and PCV2d in support of this construct for commercial vaccines.

The following examples are include for the sake of completeness of disclosure and to illustrate the methods of making the compositions and composites of the present invention as well as to present certain characteristics of the compositions. In no way are these examples intended to limit the scope or teaching of this disclosure.

### Example 1

### Generation and Testing of Chimeric Vaccines

In one embodiment, shuffled PCV2 capsid genes were first cloned into the backbone of PCV2a. A total of four chimeric viruses were found viable in vitro and subsequently used to infect pigs to assess their ability to induce cross-neutralizing antibodies against different PCV2 subtypes. One chimeric virus (PCV2-3cl.14) induced higher neutralizing antibody titers against different PCV2 subtypes. A candidate vaccine (PCV1-3cl.14) was produced by cloning the shuffled 3cl.14 capsid gene into the genomic backbone of the non-pathogenic PCV1.

A vaccine efficacy study conducted in thirty two (32) pigs revealed that the chimeric virus PCV1-3cl.14 induces protective immunity against challenge with PCV2b or PCV2d. Pigs vaccinated with PCV1-3cl.14 and subsequently challenged with either PCV2b or PCV2d had significantly decreased microscopic lesions and lower viral DNA loads in serum and lymphoid tissues compared to mock-vaccinated pigs. The chimeric PCV1-3cl.14 virus provides a strong candidate for a novel second generation PCV2 vaccine for a pig population commonly infected with variable PCV2 strains.

Cells:

A subclone of the PK-15 cell line that is free of PCV1 contamination was produced previously by end-point dilution of PK-15 cells (ATCC CCL-33). This subclone PK-15 cell line was cultured in Minimal Essential Medium (MEM) supplemented with 10% Fetal Bovine Serum (FBS) and antibiotics and was used in the serum virus neutralization assay and to propagate all virus stocks for this study.

DNA Shuffling of Capsid Genes from Five Different PCV2 Subtypes:

The capsid gene sequences representing each of the five genetically-diversified PCV2 subtypes were selected for DNA shuffling, including PCV2a (strain 40895, GenBank accession number AF264042, SEQ ID NO: 9), PCV2b (strain NC16845, accession number GU799576, SEQ ID NO: 11), PCV2c (accession number EU148503, SEQ ID NO: 15), PCV2d-1 (accession number AY181947, SEQ ID NO: 13), and “PCV2e” (accession number EF524533, SEQ ID NO: 17). The PCV2a and PCV2b strains were isolated from U.S. pigs and described in Fenaux et al. and Beach et al., while the PCV2c, PCV2d-1, and “PCV2e” capsid genes were synthesized by GenScript (Piscataway, N.J.). See Fenaux M, Opriessnig T, Halbur P G, Elvinger F, Meng X J. “A chimeric porcine circovirus (PCV) with the immunogenic capsid gene of the pathogenic PCV type 2 (PCV2) cloned into the genomic backbone of the nonpathogenic PCV1 induces protective immunity against PCV2 infection in pigs” J Virol 78 (2004) 6297-6303 and Beach N M, Ramamoorthy S, Opriessnig T, Wu S Q, Meng X J. “Novel chimeric porcine circovirus (PCV) with the capsid gene of the emerging PCV2b subtype cloned in the genomic backbone of the non-pathogenic PCV1 is attenuated in vivo and induces protective and cross-protective immunity against PCV2b and PCV2a subtypes in pigs” Vaccine 29 (2010) 221-232.

DNA shuffling was used to shuffle the five different PCV2 capsid genes essentially as previously described for PRRSV in Ni Y Y et al. See Ni Y Y, Opriessnig T, Zhou L, Cao D, Huang Y W, Halbur P G, Meng X J. “Attenuation of porcine reproductive and respiratory syndrome virus by molecular breeding of virus envelope genes from genetically divergent strains” J Virol 87 (2013) 304-313. Briefly, the capsid gene DNAs from each of the five PCV2 strains were mixed in equimolar amounts with a total of 5 μg DNA and diluted in 50 μl of 50 mM Tris-HCl (pH 7.4) and 10 mM MgCl₂. The mixture was incubated at 15° C. for 3 min with 0.15 U of DNase I (Sigma). DNA fragments ranging from 50 to 150 bp in size were purified from 2% agarose gels, and subsequently added to a Pfu PCR mixture consisting of 1×Pfu buffer, 0.2 mM each deoxynucleoside triphosphate (dNTP), and 0.06 U Pfu polymerase. A PCR program without using primers (95° C. for 4 min; 40 cycles of 95° C. for 30 s, 60° C. for 30 s, 57° C. for 30 s, 54° C. for 30 s, 51° C. for 30 s, 48° C. for 30 s, 45° C. for 30 s, 42° C. for 30 s, and 72° C. for 2 min; and finally, 72° C. for 7 min) was performed to reassemble the digested DNA fragments. Subsequently, specific primers flanking the shuffled PCV2 capsid region, UniRep-F and 2aORF2-R (Table 1), were used to amplify the shuffled PCV2 capsid using Pfu Ultra II Hotstart PCR Master Mix (Agilent Technologies) per the manufacturer's instructions (95° C. for 4 min, 10 cycles of 95° C. for 30 s, 50° C. for 30 s, 72° C. for 30 s, 25 cycles of 95° C. for 30 s, 54° C. for 30 s, 72° C. for 30 s, and finally 72° C. for 7 min).

TABLE 1
Oligonucleotide primers

| | | SEQ |
|---|---|---|
| Primer | Sequence (5′→3′) | ID NO |
| uniRep_F | TTACTGAGTCTTTTTTATCACTTCGTAAT | 19 |
| | GG | |
| 2aORF2_R | CTTTCGTTTTCAGATATGACGTATCCAAG | 20 |
| | GAGGCG | |
| uniRep_R | ACCCATTACGAAGTGATAAAAAAGACTCA | 21 |
| | G | |
| SacII_uni_R | AGCCCGCGGAAATTTCTGACAAACGTTAC | 22 |
| SacII_uni_F | TTTCCGCGGGCTGGCTGAACTTTTGAAAG | 23 |
| PCV1_DSORF2_F | CTTTTTTGTTATCACATCGTAATGGTTTT | 24 |
| | TATT | |
| PCV1_DSORF2_R | TTCTTTCACTTTTATAGGATGACGTATCC | 25 |
| | AAGGA | |
| PCV1_BB_F | CCTCCTTGGATACGTCATCCTATAAAACT | 26 |
| | GAAAGAA | |
| PCV1_BB_R | AAATAAAAACCATTACGATGTGATAACAA | 27 |
| | AAAAG | |
| NB-56-m2b | GAGGTGTTCGGCCCTCCTCA | 28 |
| PCV2-83F | AAAAGCAAATGGGCTGCTAA | 29 |
| PCV2-83R | TGGTAACCATCCCACCACTT | 30 |
| PCV1 qRepF | TGGAGAAGAAGTTGTTGT | 31 |
| PCV1 qRepR | TCTACAGTCAATGGATACC | 32 |
| M13(-20)F | GTAAAACGACGGCCAG | 38 |
| M13R | CAGGAAACAGCTATGAC | 39 |

Construction of Infectious DNA Clones of Chimeric PCV2a and PCV1 Viruses with Shuffled PCV2 Capsid Genes:

The shuffled capsid gene product libraries were cloned into the blunt end cloning vector, pCR-Blunt II, using the Zero Blunt® TOPO® PCR Cloning kit (Life Technologies, Carlsbad), per manufacturer's instructions. Selected clones were sequenced and analyzed for DNA shuffling efficiency, and well-shuffled capsid genes containing regions from all 5 PCV2 subtypes were amplified and subsequently cloned into the infectious DNA clone backbone of the PCV2a strain 40895 by fusion PCR, essentially described in Opriessnig T, Xiao C T, Gerber P F, Halbur P G, Matzinger S R, Meng X J. “Mutant USA strain of porcine circovirus type 2 (mPCV2) exhibits similar virulence to the classical PCV2a and PCV2b strains in caesarean-derived, colostrum-deprived pigs” J Gen Virol 95 (2014) 2495-2503.

Briefly, the shuffled PCV2 capsids were amplified using primers UniRep-F and 2aORF2-R (Table 1). The PCV2a infectious DNA clone backbone sequence was amplified in two fragments that flank the PCV2 capsid region using primers SacII-uni-F and UniRep-R, and primers 2aORF2F and SacII-uni-R, for PCV2a fragments 1 and 2, respectively (Table 1). All three PCR reactions were performed using ACCUZYME MIX™ (Bioline) at 95° C. 10 min, 35 cycles of 95° C. for 30 s, 54° C. for 30 s, and 68° C. for 1.5 min. The first fusion PCR was performed with the PCV2 fragment 1 and the shuffled PCV2 capsid sequence using the external primers SacII-uni-F and 2aORF2-R. Subsequently, a second fusion PCR reaction was performed with the product of the first fusion PCR reaction and the PCV2a fragment 2, using the external primers SacII-uni-F and SacII-uni-R (Table 1). All fusion PCR reactions were performed using ACCUZYME MIX™ at 95° C. 10 min, 35 cycles of 95° C. for 30 s, 60° C. for 30 s, and 68° C. for 4 min. The full-length chimeric PCV2a containing each individual shuffled PCV2 capsid was amplified, and cloned into the pCR-Blunt II TOPO plasmid using the Zero Blunt® cloning kit to produce infectious DNA clones of chimeric PCV2a with shuffled capsid genes.

The shuffled PCV2 capsid 3cl14 (SEQ ID NO: 7) was cloned into the infectious DNA clone backbone of the non-pathogenic PCV1 to create the vaccine candidate PCV1-3cl14 (SEQ ID NO: 37) by a similar fusion PCR protocol. Briefly, the shuffled PCV2 capsid 3cl14 sequence was amplified using primers PCV1-BB-F and PCV1-DS-ORF2-R (Table 1). The infectious DNA clone PCV1 backbone sequence was amplified from the PBSK+plasmid containing PCV1 in two fragments that flank the PCV1 capsid region using primers M13F (−20) and PCV—BB—R, and primers PCV-DS-ORF2-F and M13R, for PCV1 fragments 1 and 2, respectively (Table 1). All three PCR reactions were performed using Platinum® PCR Supermix (Thermo Scientific) at 94° C. 3 min, 35 cycles of 94° C. for 30 s, 55° C. for 30 s, and 68° C. for 1 min. Fusion PCR was performed first with the PCV1 fragment 1 and the shuffled PCV2 capsid 3cl14 fragment using the external primers M13F and PCV1-DS-ORF2-R (Table 1). A second fusion PCR reaction was performed with the product of the first fusion PCR reaction and PCV1 fragment 2, using the external primers M13F and M13R (well known commercially available pUC/M13 sequencing primers). The full-length chimeric PCV1 virus containing the shuffled capsid 3cl14 was cloned into pCR-Blunt II TOPO using the Zero Blunt® cloning kit to produce the infectious DNA clone of vaccine candidate chimeric PCV1 virus 3cl14.

Preparation of Virus Stocks:

The infectious virus stocks of PCV2b strain NC16845, U.S. PCV2d-2 strain JX535296, and each of the PCV2a capsid-shuffled chimeric viruses were produced by transfecting PK-15 cells with concatemerized viral genomes from the respective infectious DNA clones. Briefly, the respective PCV2 genomes were excised from pCR-Blunt II TOPO by SacII digestion, concatemerized, and transfected into PK-15 cells to determine the viability and infectivity by immunofluorescence assay (IFA). The virus stocks for the chimeric PCV1-2a and chimeric PCV1 containing shuffled 3cl14 capsid (PCV1-3cl14) were prepared similarly as described above except that the viral genome was excised from the pCR-Blunt II TOPO vector by digestion with KpnI prior to concatemerization.

Determination of the Infectivity and Cross-Neutralizing Activities of the PCV2 Capsid-Shuffled Chimeric Viruses:

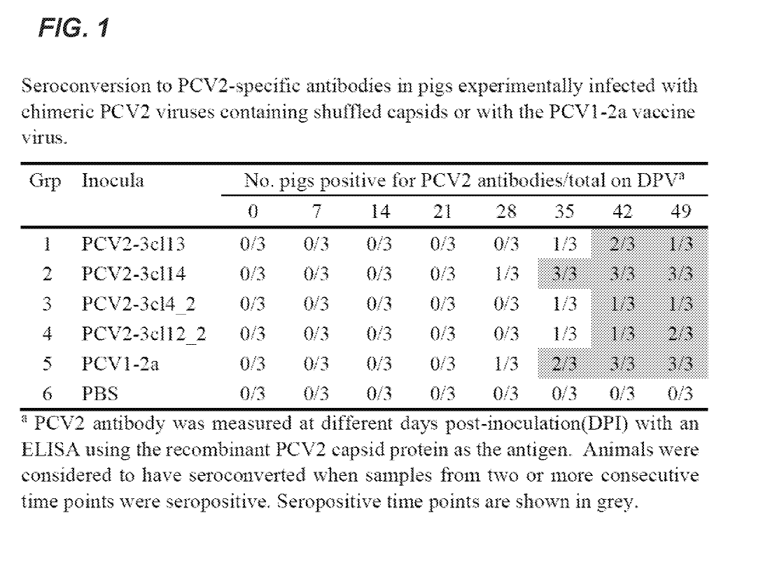

To initially identify viable shuffled chimeric viruses with improved cross-neutralizing activities against different PCV2 subtypes, a pilot pig infection study was first conducted with a limited number of animals (n=3). A total of eighteen (18), 4-week-old, cross-breed conventional pigs were purchased from a commercial farm that is known to be free of PRRSV and M. hyopneumoniae without active PCV2 circulation as determined by regular PCV2 PCR on selected batches of pigs. Sows have low amounts of antibodies against PCV2 or are seronegative and litters from negative sows without cross-fostering were selected. The piglets were randomly assigned to six groups of three pigs each, and each group of pigs was housed separately. Prior to inoculation, each pig was weighed, bled, and confirmed to be negative for PCV2 by PCR and serology. Five groups were inoculated intramuscularly each with 5 ml (103.66 TCID50/mL) of either chimeric virus PCV1-2a or one of the four PCV2 capsid-shuffled chimeric viruses (PCV2-3cl13, PCV2-3cl14, PCV2-3cl4-2, or PCV2-3cl12-2). One group was mock-inoculated similarly with 5 mL of PBS buffer (FIG. 1). Blood was collected weekly, and animals were monitored for seroconversion to PCV2 antibodies by ELISA and evidence of PCV2 infection by qPCR. Animals were necropsied at fifty-six (56) days post-infection (dpi). The weekly serum samples were used to perform serum virus neutralization test against strains representing different PCV2 subtypes.

Serum Virus Neutralization Assay:

Serum samples collected from infected pigs were tested for neutralizing antibody titers against the wild-type PCV2a, PCV2b, PCV2d-1, and PCV2d-2 strains by IFA. Briefly, the serum samples were serially diluted 1:2 in PBS and mixed with 150 TCID50 of PCV2a, PCV2b, PCV2d-1, or PCV2d-2 virus stocks, respectively, at an equal volume ratio and incubated for 1 hr at 37° C. The serum-virus mixture was then added to PK-15 cells in a 96 well plate in duplicate. After 72 hrs incubation at 37° C., an IFA was performed using pig sera against PCV2a diluted 1:1000, as the primary antibody and FITC-conjugated goat anti-pig IgG (KPL) diluted 1:50 as the secondary antibody. The 50% serum neutralizing antibody titers were determined as the highest dilution at which there was 50% or greater reduction in virus titer compared with the average of the serum from PBS control pig group at that dilution.

Vaccination Efficacy and Challenge Study in Conventional Pigs:

The chimeric virus containing shuffled capsid 3cl14 in the backbone of PCV2a induced significantly higher neutralizing antibody responses against different PCV2 strains. Therefore, the shuffled capsid sequence 3cl14 was subsequently cloned into the infectious DNA clone backbone of non-pathogenic PCV1 to produce a PCV1-3cl.14 chimeric virus as the vaccine candidate. Subsequently, a pig challenge study was conducted to evaluate the efficacy of the candidate PCV1-3cl.14 chimeric virus vaccine against infection with currently predominant circulating PCV2b as well as the emerging PCV2d-2.

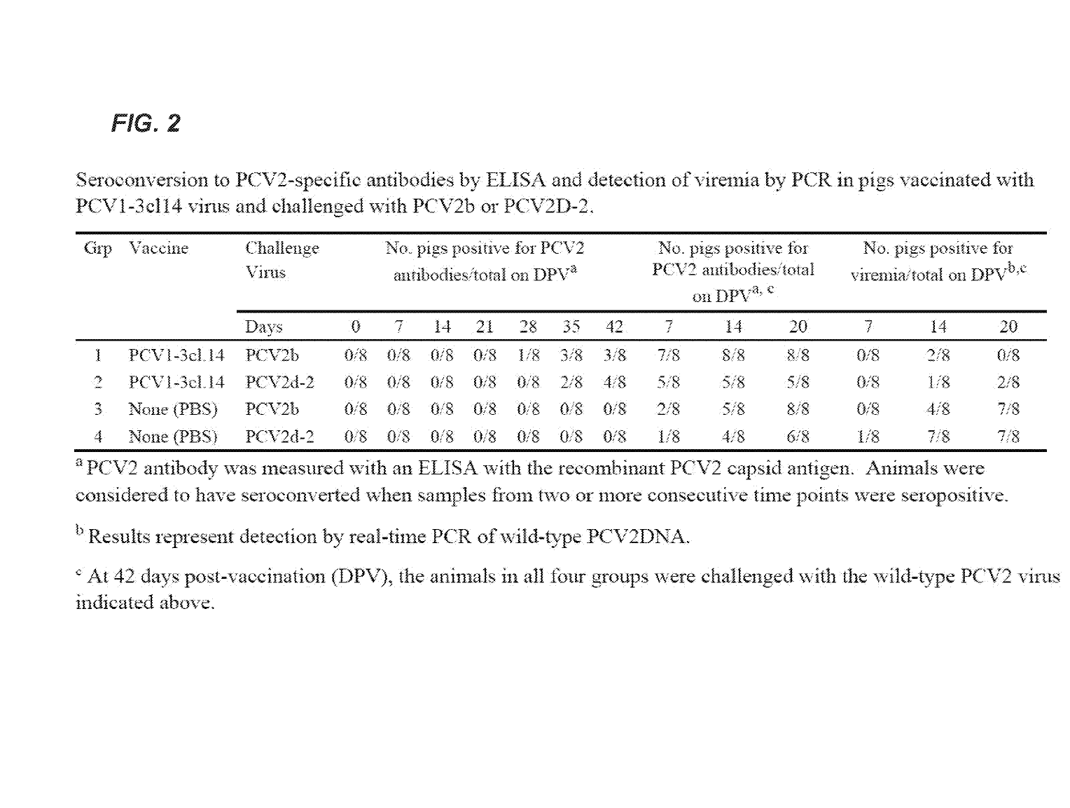

Briefly, a total of thirty-two (32), 3-week-old, cross-breed conventional pigs were purchased from a commercial farm that is known to be free of PRRSV and M. hyopneumoniae, and is negative for PCV2. The animal study was approved by Iowa State University IACUC as well as by Virginia Tech IACUC. The piglets were randomly assigned to four groups of eight pigs each. Prior to inoculation, each pig was weighed, bled, and confirmed to be negative for PCV2. Groups one (1) and two (2) pigs were each vaccinated intramuscularly (IM) in the neck region with 5 ml of the candidate PCV1-3cl.14 chimeric virus vaccine (103.7 TCID50/mL per pig). Groups three (3) and four (4) pigs were each mock-vaccinated IM with 5 ml PBS buffer (FIG. 2). All animals were monitored daily for clinical signs including wasting, respiratory distress, and behavioral changes such as lethargy and inappetence. Blood samples were collected prior to inoculation, and weekly thereafter from each pig through forty-two (42) days post-vaccination (dpv).

At forty-two (42) dpv, groups one (1) (vaccinated) and three (3) (mock-vaccinated) pigs were each challenged with 104.8 TCID50 (2.5 ml intranasally and 2.5 ml IM) of the PCV2b NC16845 virus strain, and groups two (2) (vaccinated) and four (4) (unvaccinated) were each similarly challenged with 104.8 TCID50 of the PCV2d-2 JX535296 virus strain. Blood samples were collected weekly through twenty (20) days post-challenge (dpc) (or sixty-two (62) dpv), at which time all pigs were weighed and necropsied. A panel of serum and tissue samples was collected for quantification of viral DNA loads and for histological examination of PCV2-associated lesions.

Gross Pathology and Histopathology Evaluation:

Necropsies were performed at twenty (20) days post challenge (dpc) on all pigs in a to the treatment status blinded fashion. Estimates of macroscopic lung lesions (ranging from 0 to 100% of the lung affected) and lymph node size (ranging from zero (0) [normal] to three (3) [four times the normal size]) were obtained for each pig. Sections of lung, lymph nodes (superficial inguinal, mediastinal, tracheobronchial, and mesenteric), tonsil, heart, thymus, kidney, spleen, and liver were collected during necropsy and processed routinely for histological examination and PCV2 immunohistochemistry (IHC) (Iowa State University Veterinary Diagnostic Lab). Also, samples of tracheobronchial lymph node (TBLN) were collected from each pig for DNA extraction and quantification of PCV2 viral genomes by real-time quantitative PCR. Microscopic lesions in the lymphoid tissues, lungs, heart, liver, kidney, ileum, and colon were scored in a treatment status blinded manner. Specifically, lymph nodes, spleen, and tonsil were evaluated for presence and degree of lymphoid depletion and histiocytic replacement.

Quantitative PCR to Quantify Viral DNA Loads in Serum and Tissues:

For both animal experiments we used a previously published protocol to extract DNA from serum and lymph node samples and a previously published qPCR SYBR green assay to quantify viral loads in these samples. For the pilot infection study (FIG. 1) and for the challenge experiment (FIG. 2), PCV2 specific primers were used to amplify a conserved region spanning the origin of replication and a portion of the replicase gene, as previously reported, using primers PCV2-83F and PCV2-83R (Table 1). For the detection of the PCV1-3cl.14 vaccine strain in the challenge study (FIG. 2), primers PCV1-qRepF and PCV1-qRepR primers (Table 1) were used to amplify only the PCV1 backbone based vaccine virus DNA.

Serology:

A PCV2-specific ELISA (Iowa State University Veterinary Diagnostic Lab) was used to detect anti-PCV2 ORF2 IgG in each serum sample as previously described. See Nawagitgul P, et al. “Modified indirect porcine circovirus (PCV) type 2-based and recombinant capsid protein (ORF2)-based enzyme-linked immunosorbent assays for detection of antibodies to PCV” Clin Diagn Lab Immunol 9 (2002) 33-40.

Sequence Confirmation of Virus Recovered from Infected Pigs:

DNA extracts from serum samples collected at twenty (20) dpc from selected pigs in each group were tested by PCR for PCV2 capsid sequences, and the amplified PCR products were sequenced to verify that the virus recovered from the infected pigs was the same virus inoculated into the animals. PCR primers Unirep-F and 2aORF-2 were used to amplify the PCV2 capsid gene in these samples using the same PCR program as described above for cloning (Table 1). Additionally, DNA extracts of TBLN tissues from selected pigs in each group were also tested to confirm that the virus detected by PCR from infected pigs was the same virus that was inoculated into the animals. PCV2b was amplified and sequenced using primers specific for PCV2b as previously described. (Beach et al. supra). The PCV2d-2 vDNA was amplified and sequenced using the same forward primer as for PCV2b and a PCV2d-specific reverse primer NB-56-m2b (Table 1).

Statistical Analysis:

Statistical analysis was performed using Prism v6.0 (Graphpad, La Jolla Calif.). A one-tailed t-test was used to analyze statistical significance between two groups, while a one-way ANOVA and then t-tests corrected for multiple comparisons were used to determine significance between three or more groups.

Generation of Infectious Chimeric Viruses Containing the Shuffled Capsid from 5 Genetically Distinct PCV2 Strains:

Traditional DNA shuffling was used to molecularly breed the capsid genes from five genetically distinct PCV2 strains representing different subtypes PCV2a PCV2b, PCV2c, and PCV2d-1, as well as “PCV2e” (divergent PCV2a) (FIG. 3). The shuffled PCV2 capsid amino acid sequences are designated in FIG. 3 as 3cl.13 (SEQ ID NO: 4), 3cl.4_2 (SEQ ID NO: 2), 3cl.14 (SEQ ID NO: 8), and 3cl.12_2 (SEQ ID NO: 6). Although the general consensus is that previously classified “PCV2e” virus isolates do not diverge enough from identified PCV2a strains to be considered their own subtype, a “PCV2e” capsid sequence was chosen to help increase the genetic diversity of the resulting shuffled capsid. The capsid gene sequences from these five strains were shuffled using DNase I digestion and reassembled by PCR without primers. A PCR product of the expected size was then generated after a second round of PCR with specific primers spanning the capsid gene. The shuffled capsid gene library was then cloned into the infectious clone backbone of PCV2a (strain 40985) to screen for viable chimeric viruses. Of the more than fifty (50) clones with “well-shuffled” capsids (containing regions from all 5 parental PCV2 strains), only four of them successfully rescued infectious virus when transfected into PK-15 cells (data not shown).

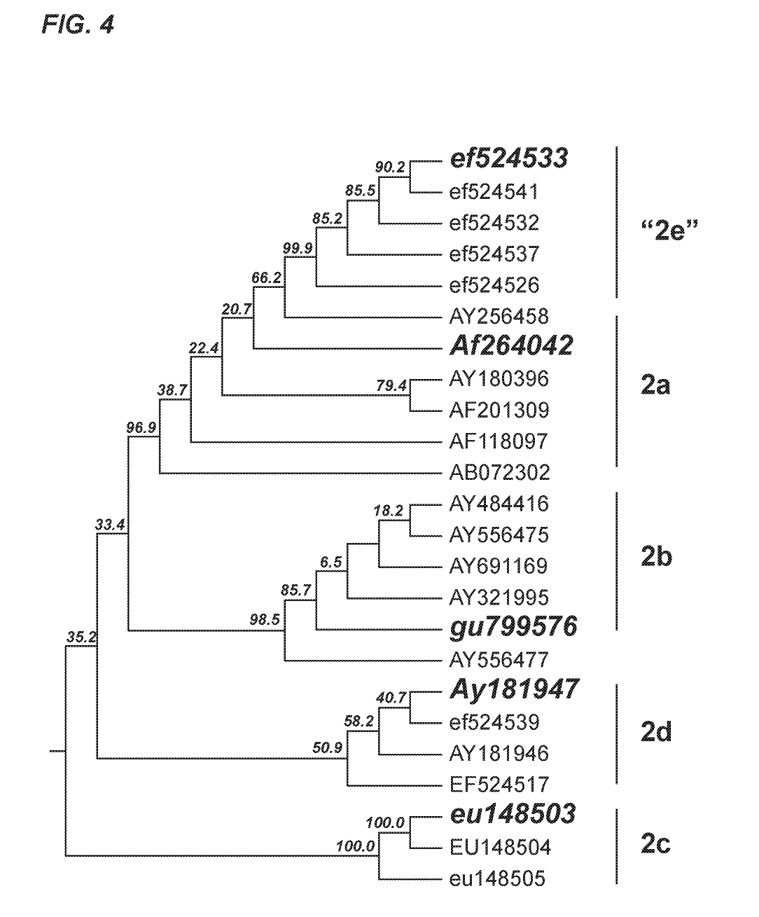

The four chimeric viruses with shuffled capsids contain a range of combinations of the genetic signatures of PCV2 genomes from all five parental strains (FIG. 3). The majority of the unique amino acid signatures introduced into the shuffled capsids originated from PCV2c. PCV2c is the most genetically distinct of the five parental strains, based on a phylogenetic analysis (FIG. 4). Therefore, it is demonstrated here that DNA shuffling successfully generated viable infectious chimeric viruses with shuffled capsid genes from 5 different PCV2 subtypes.

A Chimeric Virus PCV2-3cl14 with Shuffled Capsid Genes Induces Cross-Neutralizing Antibodies Against Different PCV2 Subtypes:

To determine the viability and screen for the best chimeric virus with shuffled capsids for subsequent challenge and efficacy study, conventional pigs were experimentally infected with each of the four chimeric viruses (PCV2-3cl.13, PCV2-3cl.14, PCV2-3cl.4_2, and PCV2-3cl.12) as well as with a previously derived chimeric PCV1-2a virus (Fenaux et al, supra). Serum samples were collected prior to infection and weekly thereafter, and all animals were monitored for seroconversion to PCV2a capsid by an ELISA (FIG. 1). All animals experimentally inoculated with PCV1-2a and/or with PCV2-3cl.14 seroconverted to PCV2 antibodies by 49 days post-inoculation (dpi), however only 2 out of 3 animals in the PCV2-3cl.12_2 and 1 of 3 pigs inoculated with either chimeric virus PCV2-3cl.13 or PCV2-3cl.4_2 were seropositive at 49 dpi (FIG. 1).

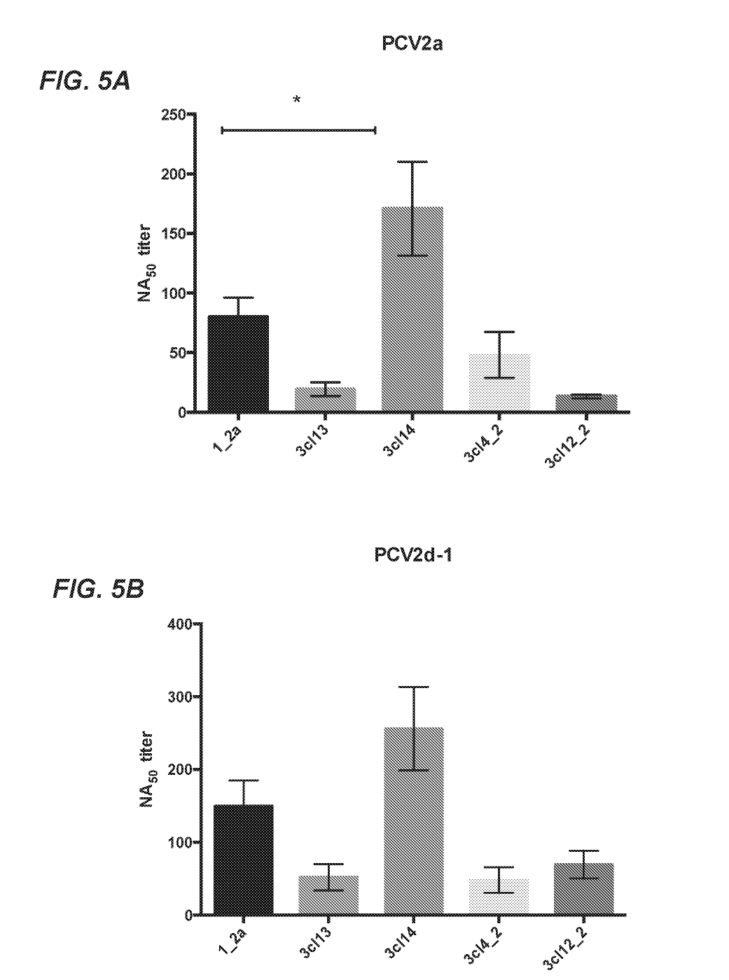

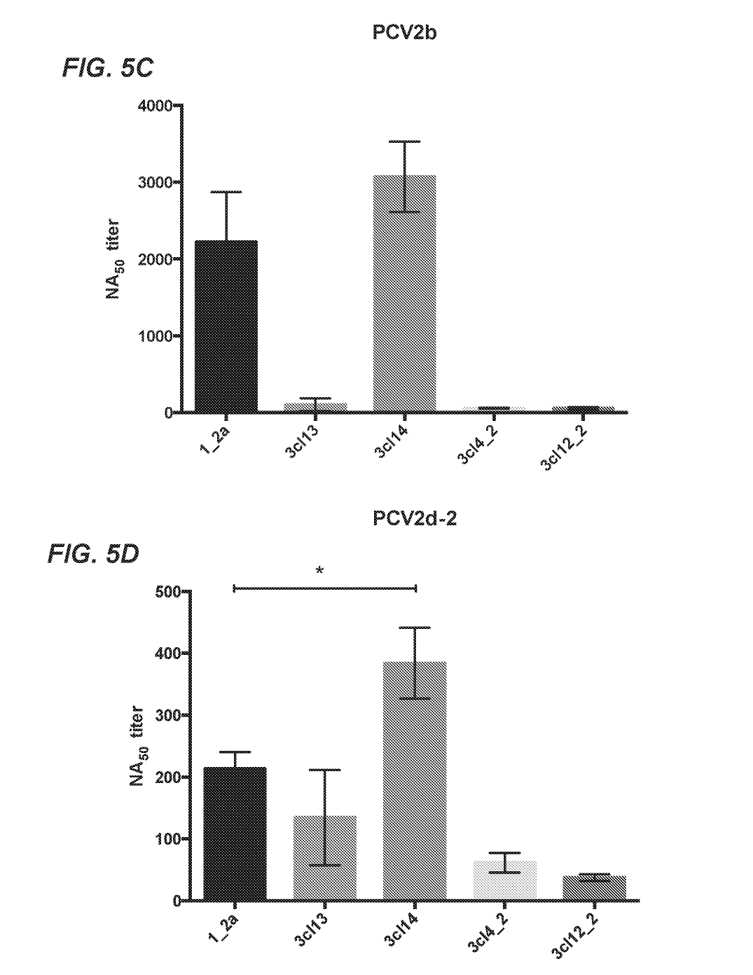

Serum samples collected from 56 dpi were tested by a serum virus neutralization assay in PK15 cells for cross-neutralizing antibodies against wild-type PCV2a (FIG. 5A), PCV2b (FIG. 5C), PCV2d-1 (FIG. 5B), and PCV2d-2 (FIG. 5D) virus strains. Infections of pigs with 3 chimeric PCV2 viruses with shuffled capsid genes (PCV2-3cl.13, PCV2-3cl.4_2, and PCV2-3cl.12) did not induce higher levels of neutralizing antibody when compared to the chimeric PCV1-2a virus which is the basis for the current Fostera™ PCV commercial vaccine. However, infection of pigs with the chimeric virus PCV2-3cl.14 with shuffled capsid genes from different PCV2 subtypes induced significantly higher neutralizing antibody titers against PCV2a and PCV2d-2 when compared to PCV1-2a (p<0.05). In addition, although not statistically significant, the chimeric virus PCV2-3cl.14 also induced higher levels of neutralizing antibody than the PCV1-2a against both PCV2b and PCV2d-2. Taken together, this pilot animal study suggests that the chimeric viruses with shuffled capsid genes are viable and infectious in pigs, and that one chimeric virus PCV2-3cl.14 induces significantly higher levels of neutralizing antibodies against genetically distinct PCV2 strains when compared to the other chimeric viruses as well as to the PCV1-2a vaccine virus. Therefore, the chimeric virus PCV2-3cl.14 was selected for the subsequent challenge and efficacy study in pigs to evaluate its potential use as a novel vaccine.

The Chimeric Virus PCV1-3cl14 Induces Protective Immunity in Conventional Pigs Against Challenge with PCV2b and PCV2d-2.

PCV2a is the genomic backbone for the chimeric virus PCV2-3cl.14. Therefore, in order to produce a novel vaccine candidate, we subsequently transferred the shuffled capsid gene from the chimeric virus PCV2-3cl.14, identified in the initial cross-neutralization study, to the genomic backbone of the non-pathogenic PCV1 to produce a new chimeric virus PCV1-3cl.14. To assess whether the chimeric virus PCV1-3cl.14 vaccine candidate protects against challenge with different PCV2 subtypes, two groups of pigs (n=8) were each vaccinated with the PCV1-3cl.14 chimeric virus, and another two groups of pigs (n=8) were mock-vaccinated with PBS as controls (FIG. 2). Blood samples were taken weekly and animals were monitored for seroconversion to PCV2 capsid antibody. At 42 days post-vaccination, one group of vaccinated and one group of mock-vaccinated animals were challenged with the predominant field strain PCV2b currently circulating in swine herds worldwide. Similarly, one vaccinated group and one mock-vaccinated group of pigs were challenged with the emerging PCV2d-2 virus. Blood samples were taken weekly after challenge and all animals were necropsied at twenty (20) dpc.

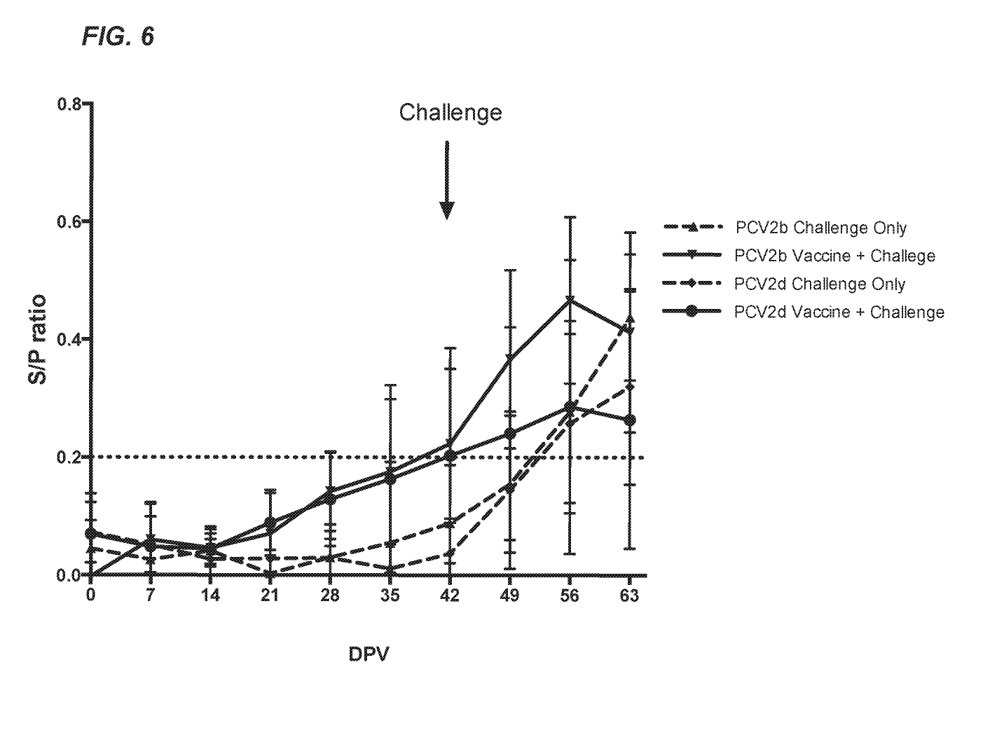

As expected, pigs in the two vaccinated groups started to seroconvert to PCV2 capsid antibody by 42 dpv, whereas mock-vaccinated groups did not seroconvert until 7-14 dpc with PCV2b or PCV2d-2 (or 49 or 56 dpv, FIG. 2, FIG. 6). A qPCR assay targeting the PCV1 replicase gene (ORF1) was used to test for PCV1-3cl.14 viral DNA from weekly sera, but PCV1-3cl.14 viral DNA was undetectable and below the detection limit of the assay in any group after vaccination (data not shown). This is consistent with previous reports of the attenuated chimeric PCV1-2 virus infections in pigs.

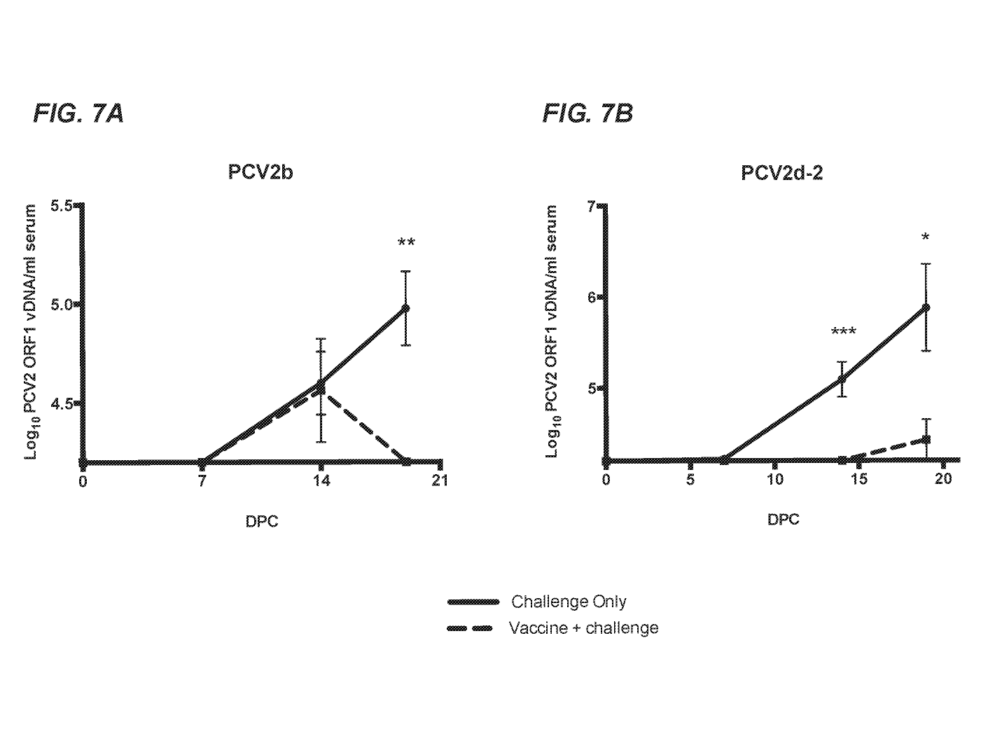

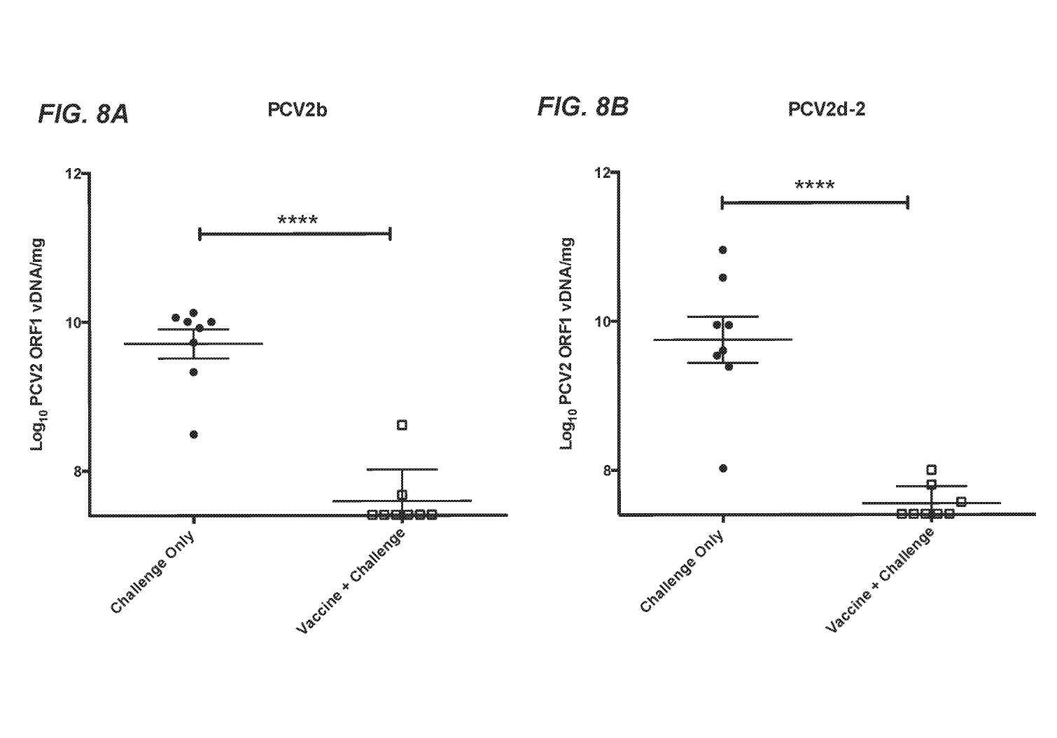

Only two out of eight animals vaccinated and subsequently challenged with PCV2b had detectable viremia, and only at fourteen (14) dpc, compared to four and seven out of eight PCV2b challenge control animals at fourteen (14) and twenty (20) dpc, respectively (FIG. 2). This difference was statistically significant, as the vaccinated and PCV2b challenged group had significantly lower levels of viral DNA loads in sera at twenty (20) dpc, compared to mock-vaccinated and PCV2b challenged animals (p<0.01) (FIG. 7A and FIG. 7B). For animals vaccinated and subsequently challenged with PCV2d-2, ⅛ at fourteen (14) dpc and 2/8 at twenty (20) dpc had detectable viremia, while ⅞ PCV2d-2 challenged control animals were positive for serum viral DNA at fourteen (14) dpc and twenty (20) dpc (FIG. 2). Also, the vaccinated and PCV2d-2 challenged group had serum viral DNA loads that were significantly reduced at fourteen (14) and twenty (20) dpc (p<0.001, p<0.05, respectively), as compared to PCV2d-2 challenge only controls (FIG. 7A and FIG. 7B). All vaccinated and subsequently challenged groups had significantly lower levels of PCV2 viremia at the peak of virus replication compared to control groups. In addition, all vaccinated and subsequently challenged groups had significantly lower levels of detectable PCV2 DNA in lymph nodes compared to mock-vaccinated and challenged groups (PCV2b=p<0.001, PCV2d-2=p<0.0001, FIG. 8A and FIG. 8B). These results indicated that vaccination with PCV1-3cl.14 chimeric virus significantly reduces the level of virus replication in pigs when challenged with the predominant PCV2b subtype or with an emerging PCV2d-2 strain.

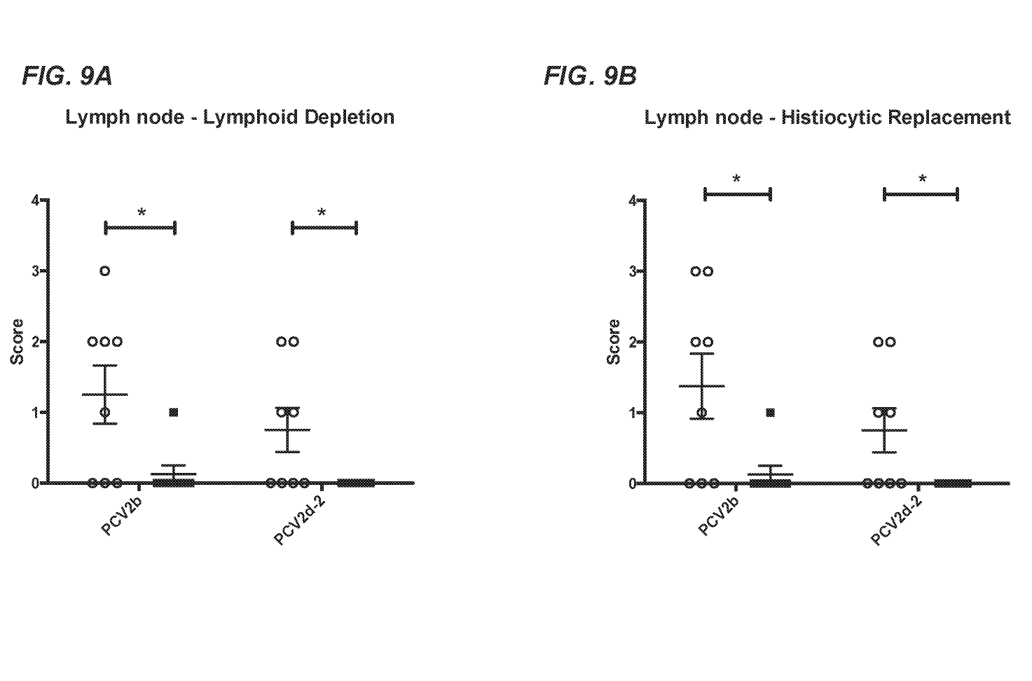

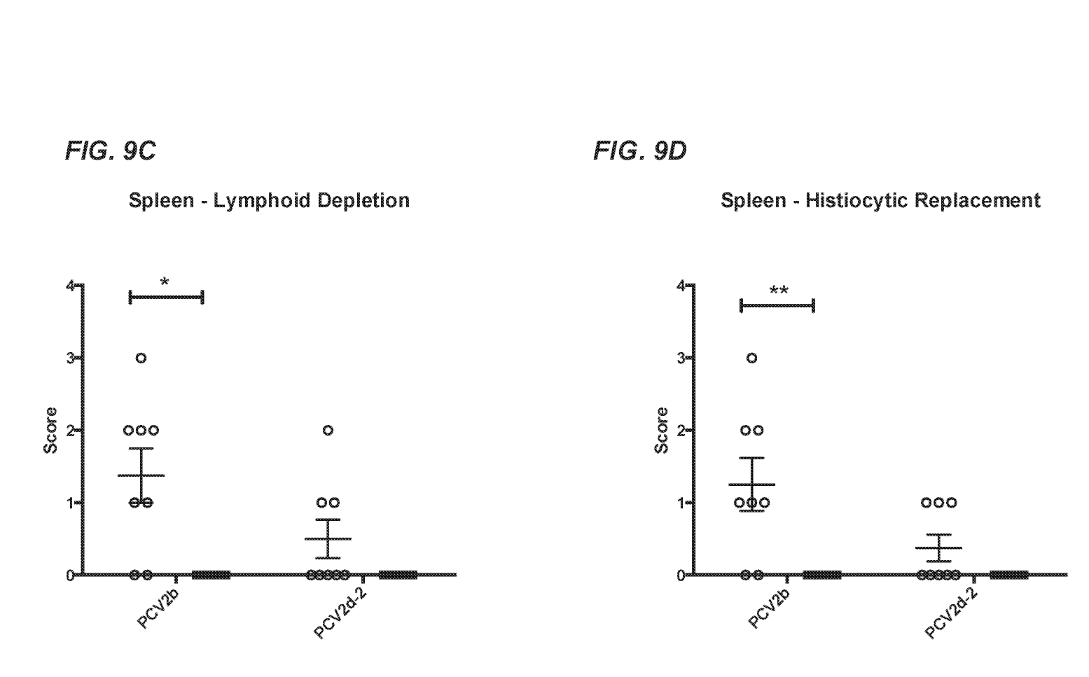

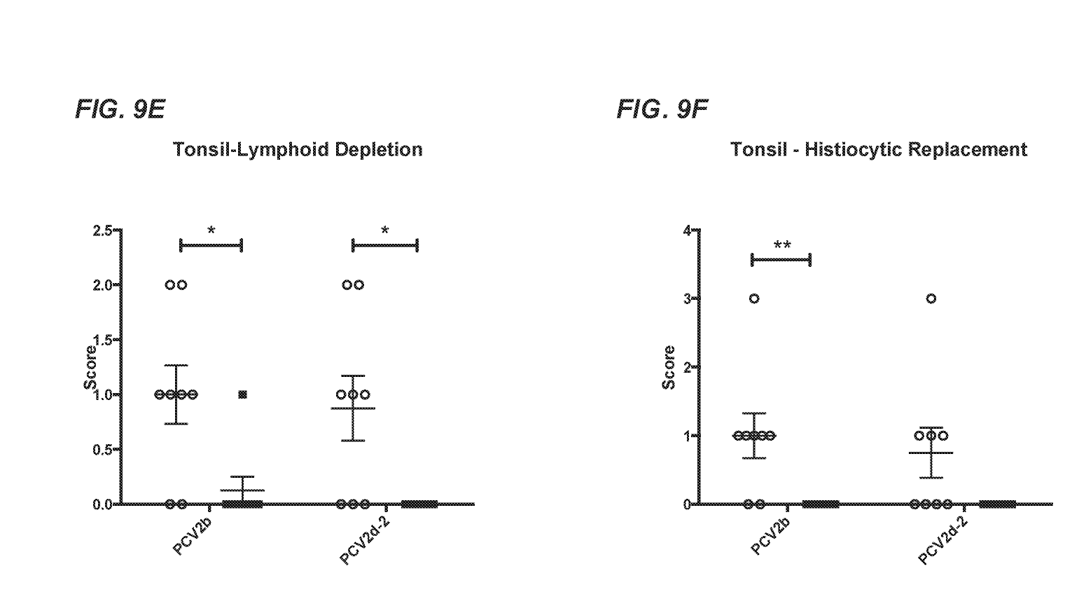

In addition to reducing viral DNA loads in sera and lymphoid tissues, vaccinated animals also had a decreased PCVAD lesion score compared to unvaccinated animals (FIG. 9A-FIG. 9F). Vaccinated pigs that were subsequently challenged with PCV2b had significantly reduced pathological lesion scores for all measures of PCVAD, which includes lymphoid depletion and histiocytic replacement in lymph nodes (FIG. 9A and FIG. 9B), spleen (FIG. 9C and FIG. 9D), and tonsil tissues (FIG. 9E and FIG. 9F), as compared to unvaccinated but PCV2b challenged controls.

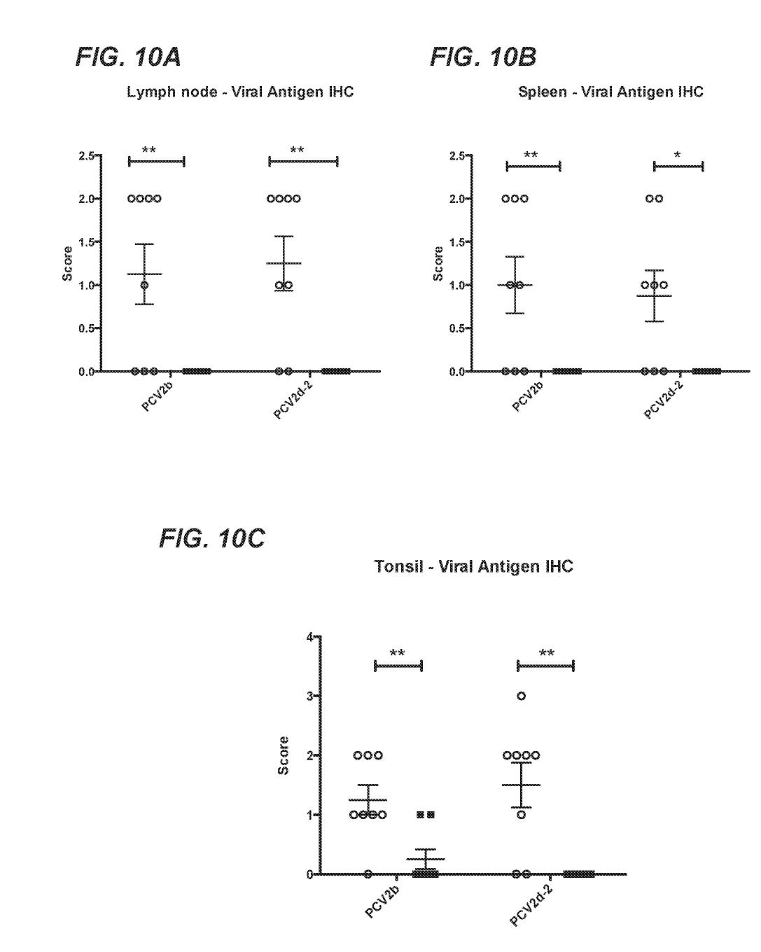

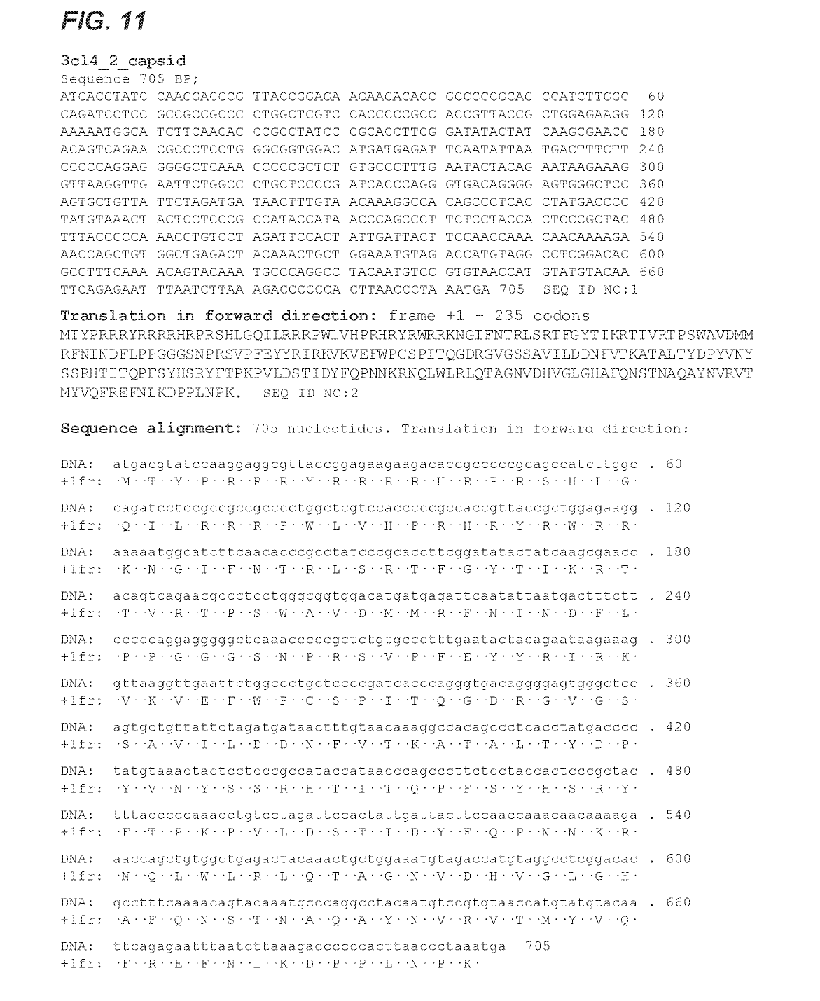

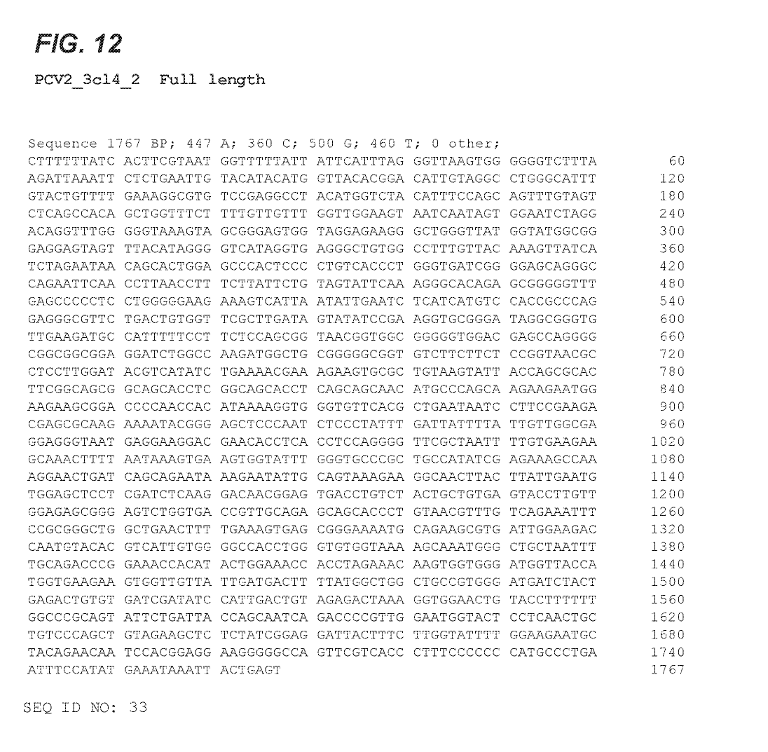

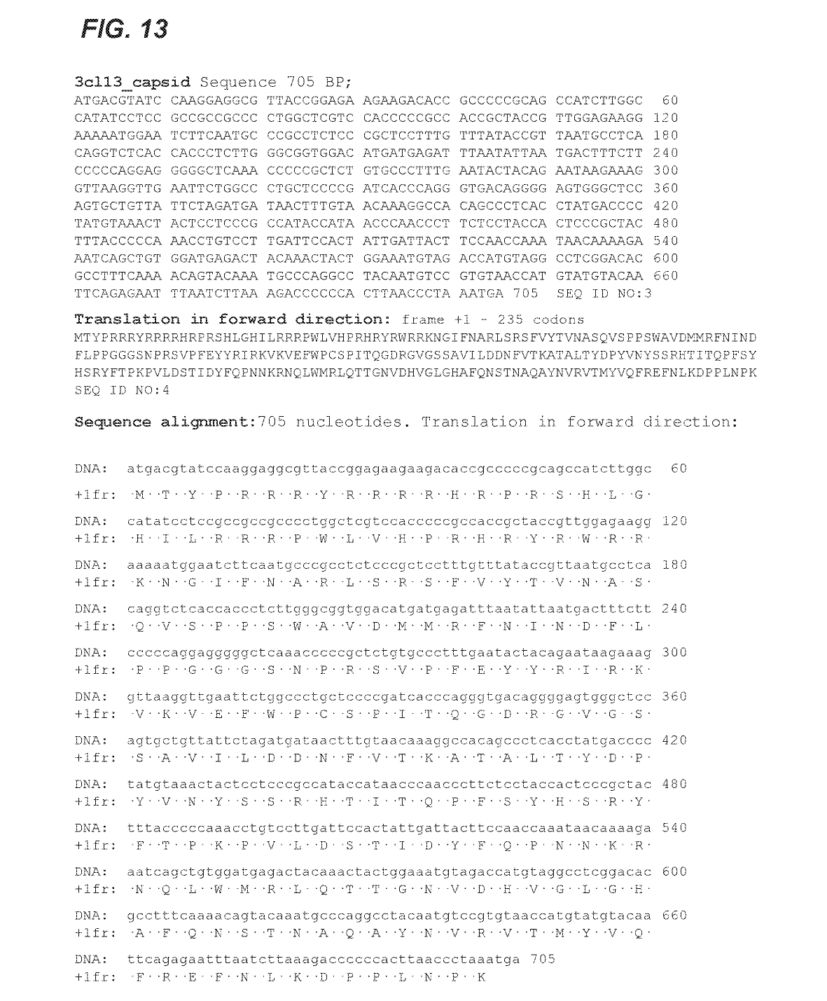

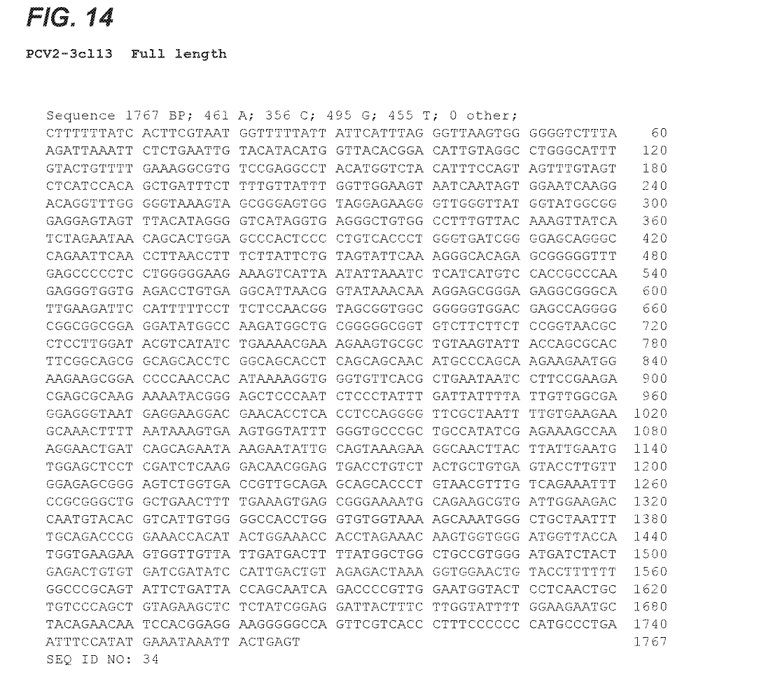

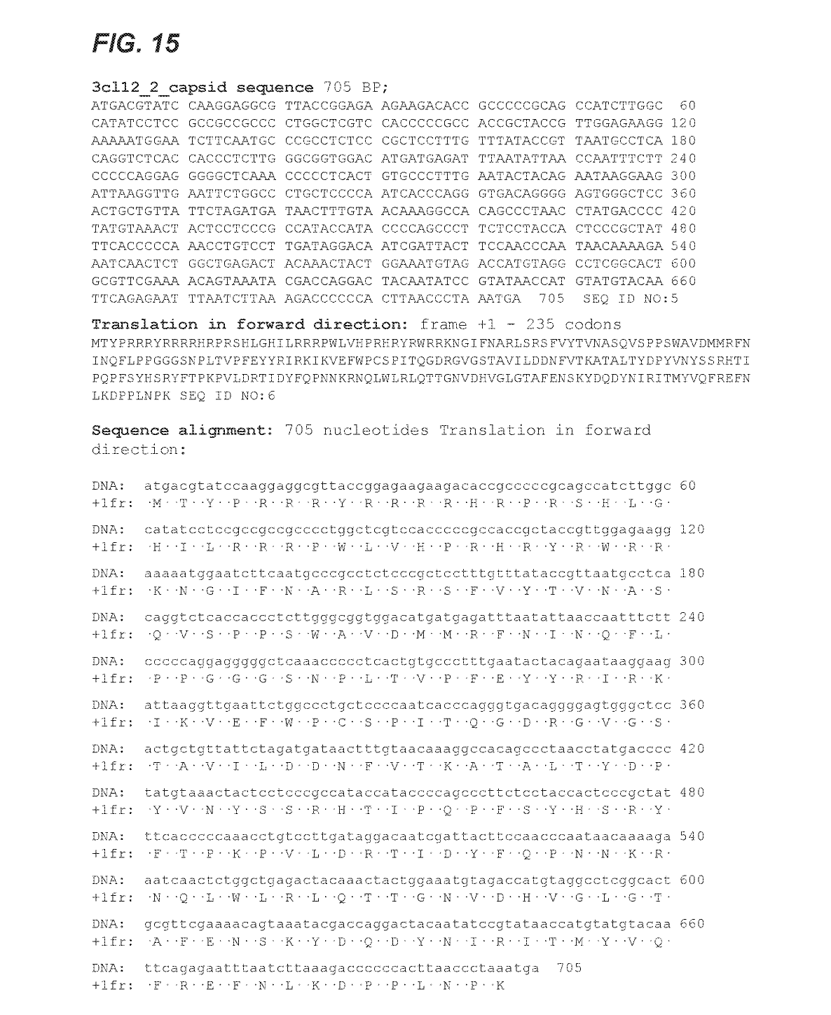

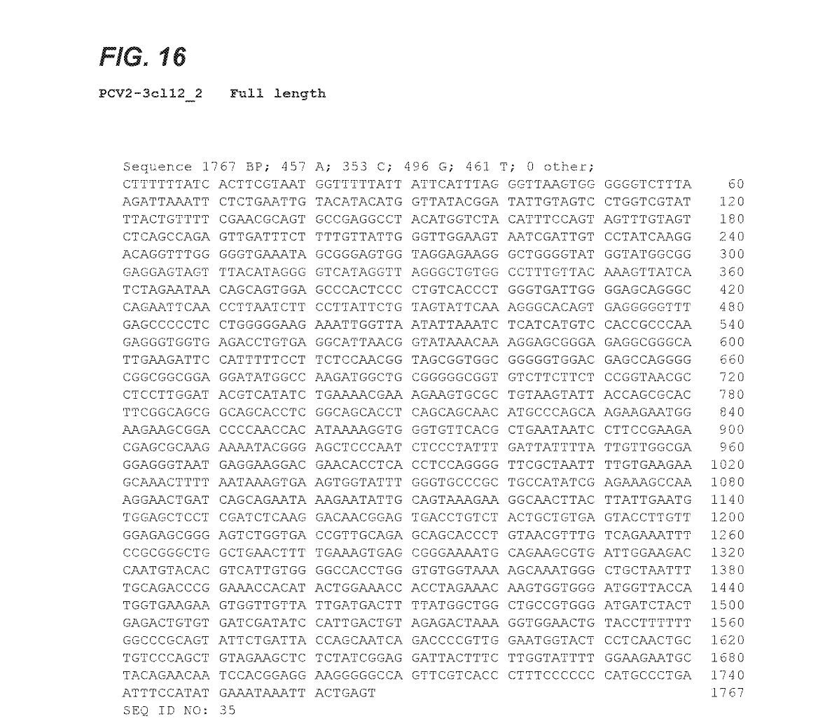

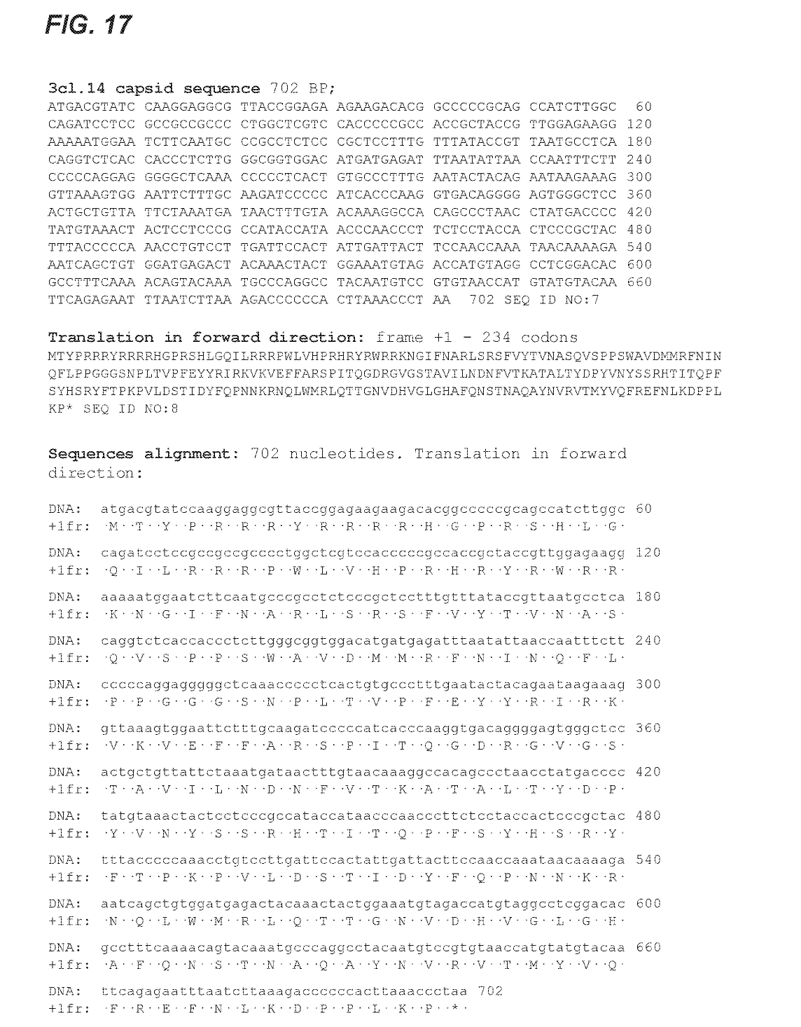

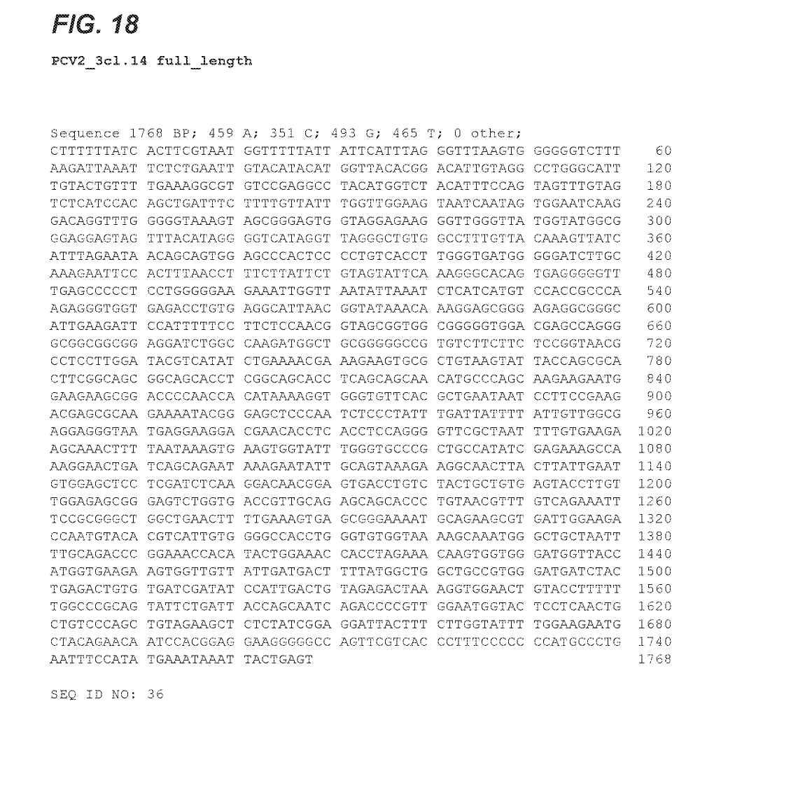

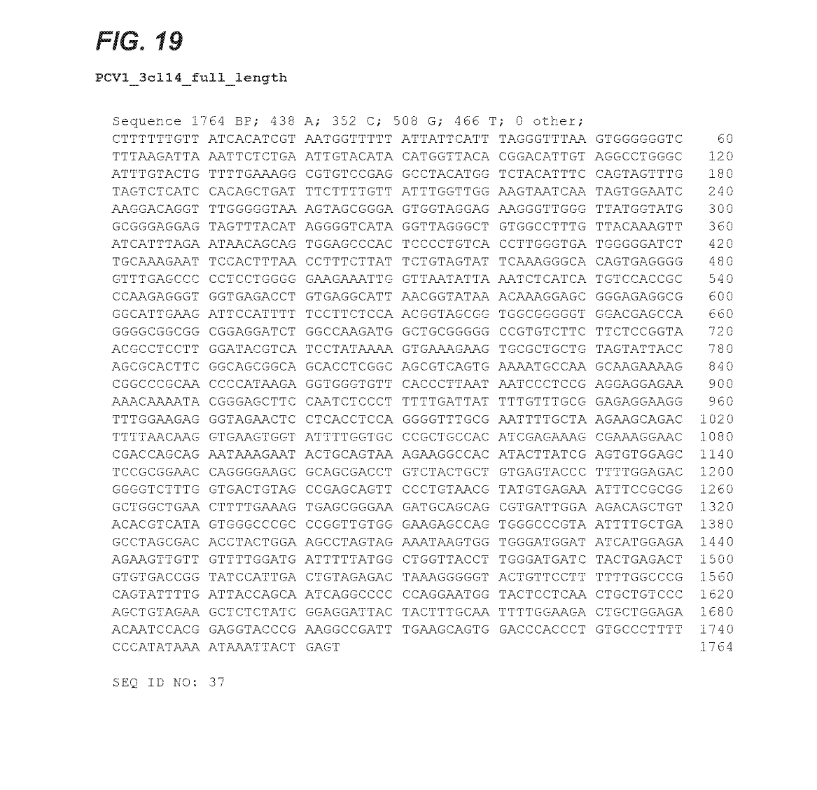

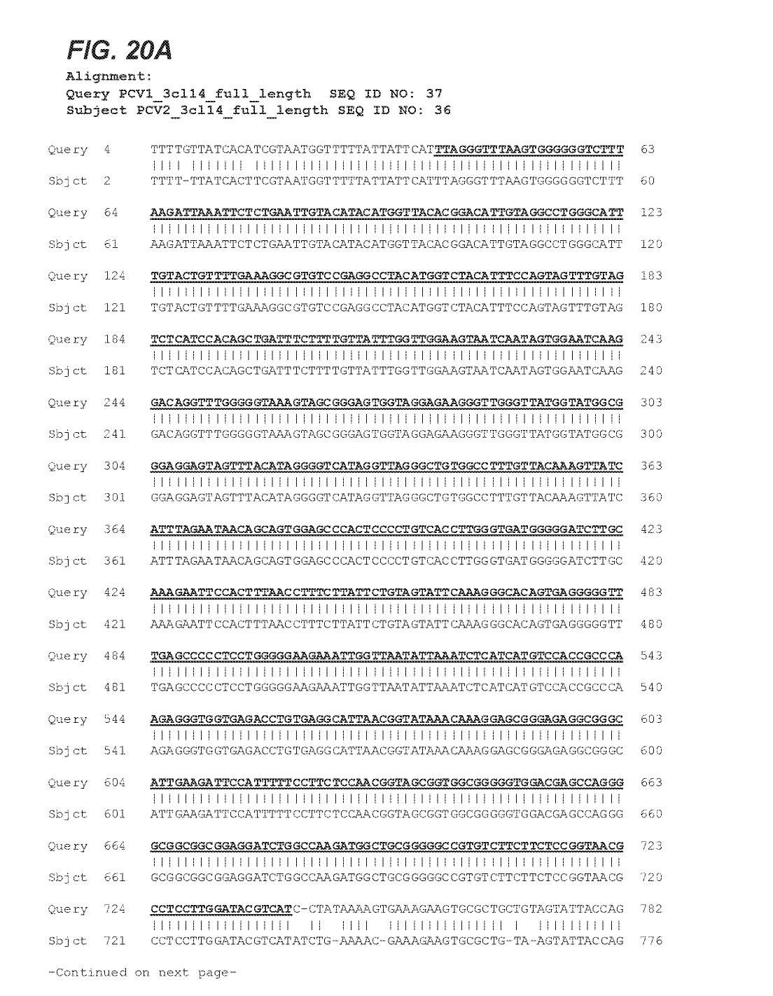

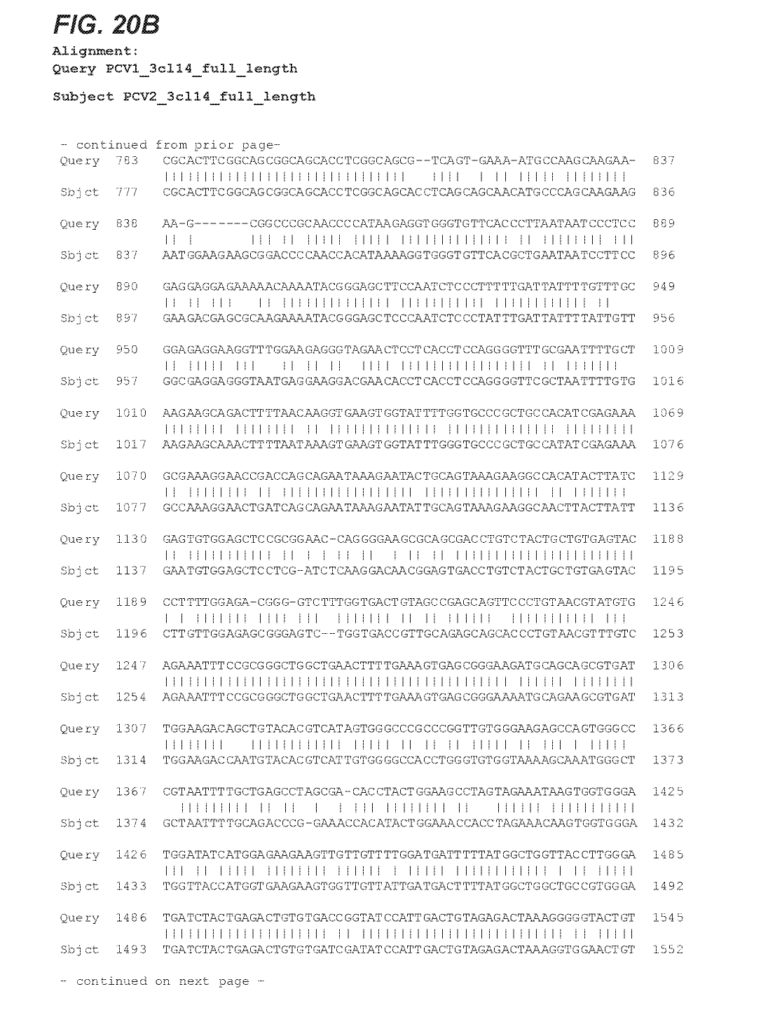

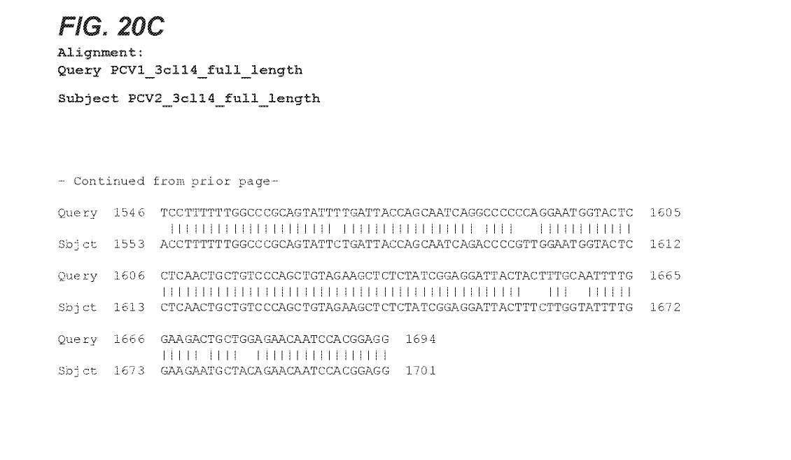

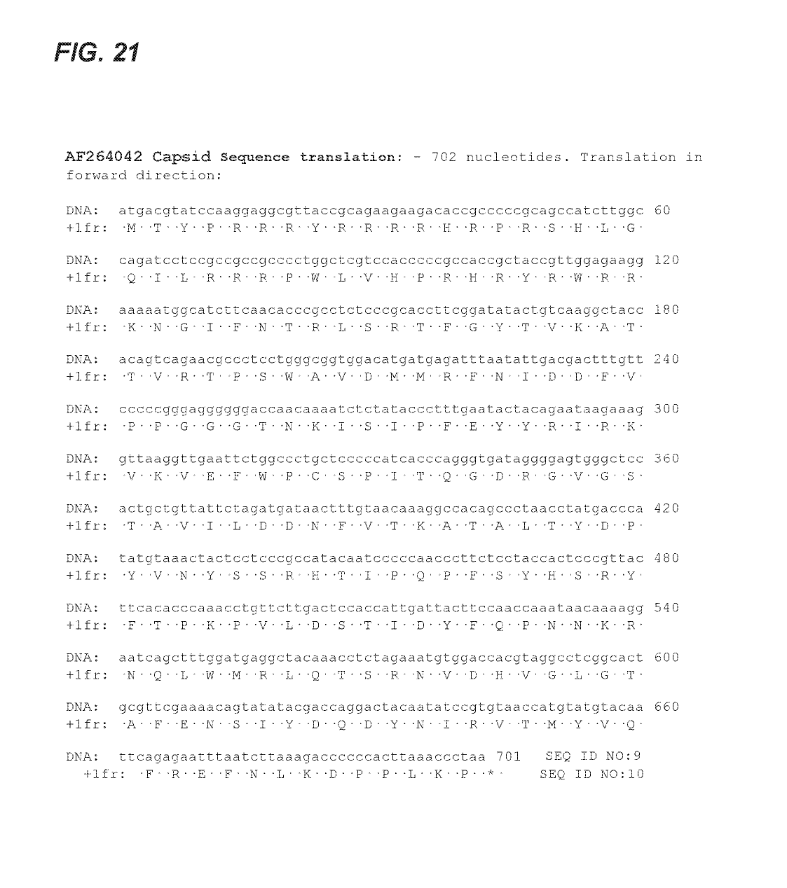

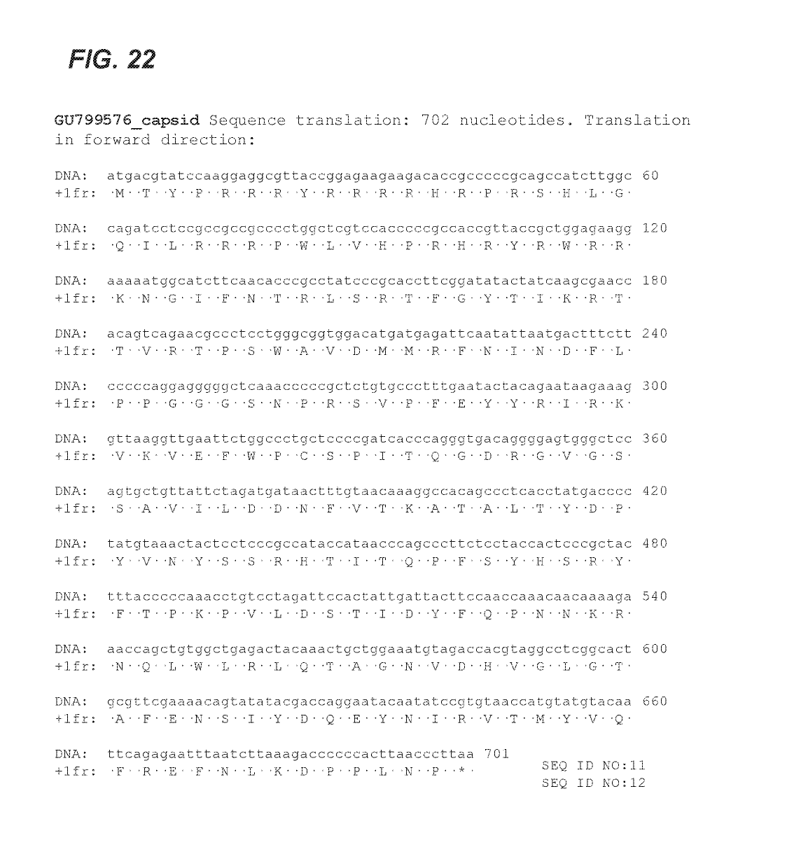

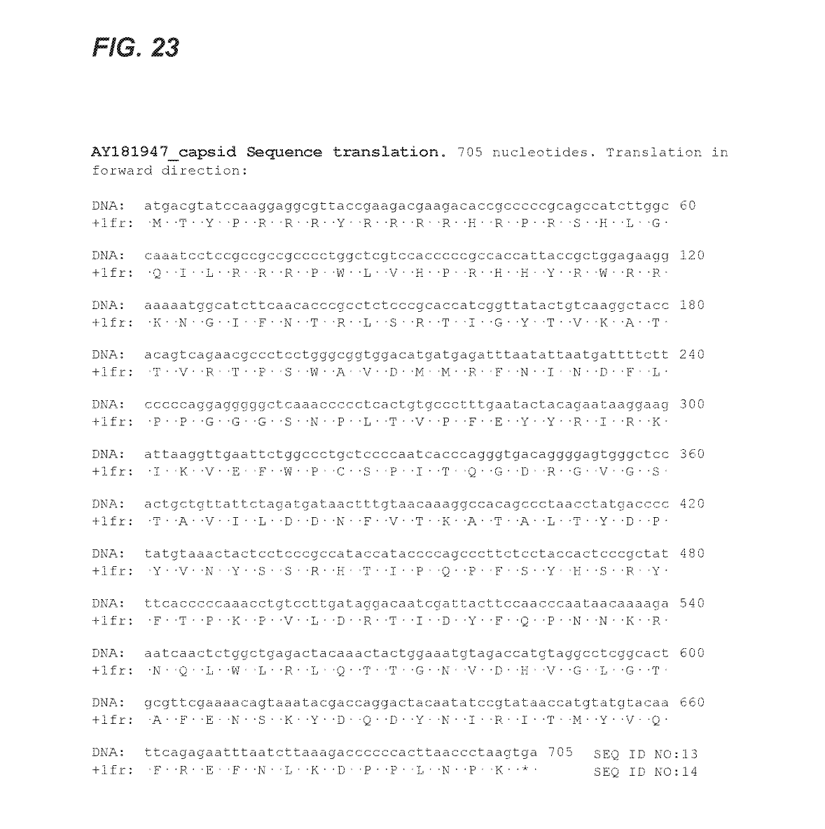

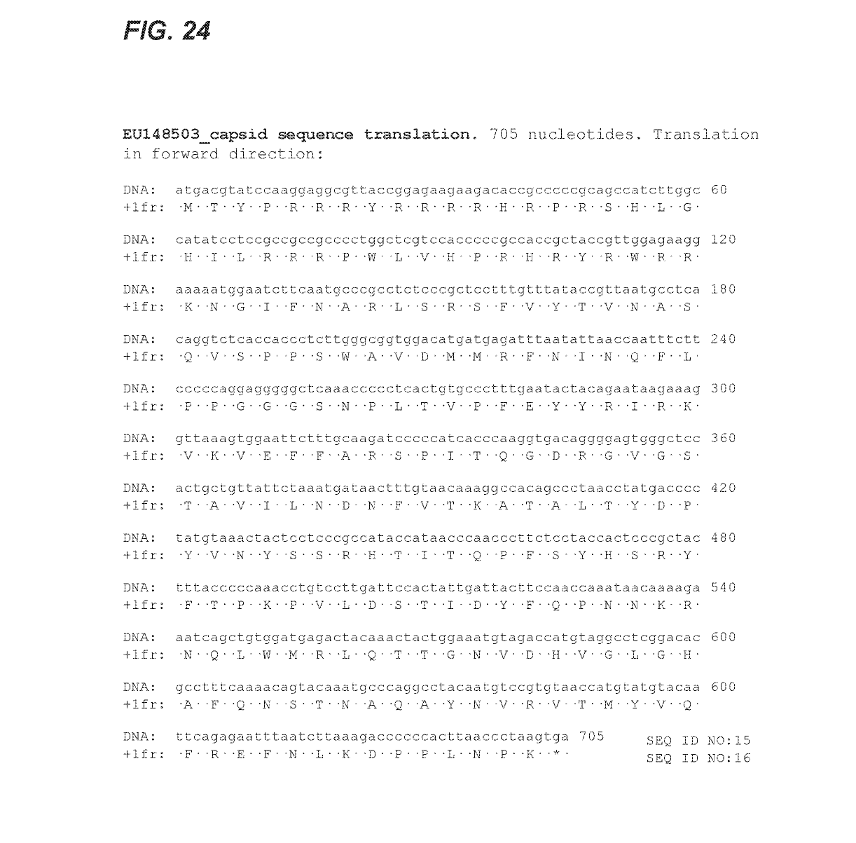

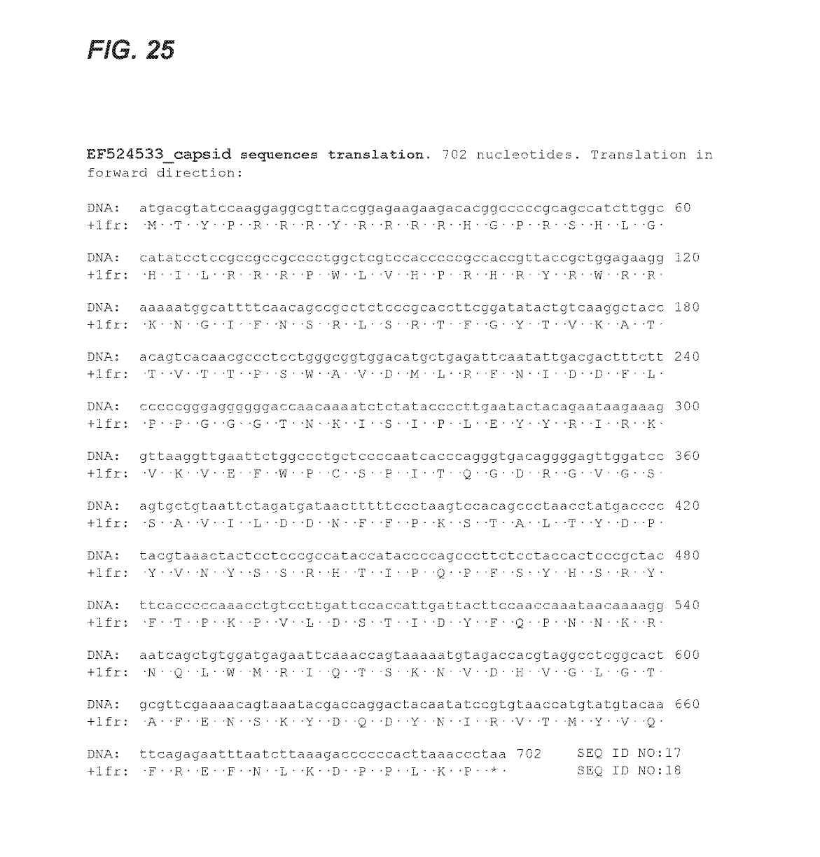

Similarly, pigs vaccinated and subsequently challenged with PCV2d-2 had significantly lower pathological lesion scores for lymph node measures, as well as tonsil lymphoid depletion (FIG. 10A, FIG. 10B, FIG. 10C) as compared to unvaccinated but PCV2d-2 challenged controls. Consistent with the results for serum and lymph node viral DNA detection, both vaccinated and subsequently challenged groups had significantly lower viral antigen scores in lymph node, spleen, and tonsil, compared to challenge only controls (FIG. 8A-FIG. 8C). Overall, these results suggest that vaccination with PCV1-3cl.14 chimeric virus vaccine candidate protects against two genetically distinct and relevant PCV2 strains, the predominant PCV2b subtype currently circulating in pig farms worldwide and the emerging PCV2d-2 strain.

### Example 2

### Chimeric Virus Derivatives

Derivatives of the chimeric PCV capsid polypeptides disclosed herein are included within the scope of this disclosure. The amino acid sequences of epitopes that are included in the chimeric PCV capsid polypeptides disclosed herein may be altered somewhat and still be suitable for use as therapeutic immunogens. For example, certain conservative amino acid substitutions may be made in epitope domains as well as other non-epitope domains of the capsid polypeptides without having a deleterious effect on the immunogenicity of included epitopes. Those of skill in the art recognize the nature of conservative substitutions, for example, substitution of a positively charged amino acid for another positively charged amino acid; substitution of a negatively charged amino acid for another negatively charged amino acid; substitution of a hydrophobic amino acid for another hydrophobic amino acid; etc. All such substitutions or alterations of the chimeric PCV capsid polypeptides disclosed herein are intended to be encompassed by the present invention, so long as included epitopes remain immunogenic. In addition, immunogens including epitopes on PCV capsid polypeptides disclosed herein need not encompass a full length PCV capsid polypeptide or epitope-containing domain. Those of skill in the art will appreciate that truncated versions of the chimeric PCV capsid polypeptides disclosed herein may be utilized or indeed preferable, so long as immunogenic epitopes remain. Derivatives of the chimeric PCV capsid polypeptides disclosed herein include truncated versions. Derivatives may include sequence modifications introduced for a variety of reasons, including or example, to eliminate or introduce a protease cleavage site, to increase or decrease solubility, to promote or discourage intra- or inter-molecular interactions such a folding, ionic interactions, salt bridges, etc, which might otherwise interfere with the presentation and accessibility of the individual epitopes along the length of the chimera. All such changes are intended to be encompassed by the present invention, so long as the resulting amino acid sequence functions to elicit a protective antibody reaction to one or more of the PCV2 types from which the epitopes originate. In general, such substituted sequences will be at least about 80 to 90% identical to the chimeric PCV capsid polypeptides disclosed herein. In certain embodiments, substituted derivative sequences will be 90 to 99% identical to the chimeric PCV capsid polypeptides disclosed herein.

As used herein reference is made to chimeric PCV capsid polypeptides and derivatives and truncated versions thereof. In certain embodiments, such may be used as isolated purified polypeptide subunit vaccines or as inactivated whole virus vaccines. The polypeptides may be chemically synthesized, or produced using recombinant DNA technology such as in bacterial, mammalian, yeast, plant or insect cells. In other embodiments however, the chimeric PCV capsid polypeptides are generated by culture of viral vectors including DNA sequences that encode chimeric PCV capsid polypeptides. The viral vector may be a PCV virus such as a PCV 1 or 2 or may be a different viral vector such as for example a pox virus or baculovirus vector. The viral vectors will encode and direct the production of the chimeric PCV capsid polypeptides that may be partially purified and used subunit vaccines or will encode and direct the production of virus particles including the chimeric PCV capsid proteins and derivatives thereof as live or killed virus vaccines.

Table 2 shows the numbers of amino acids that differ between each of the parental capsid amino acid sequences shown on FIG. 3 and each of the chimeric shuffled capsid amino acid sequences. Taking one example, as can be seen on Table 2, chimeric capsid polypeptide 3cl.14 differs from the capsid polypeptides of PCV2a and PCV2b by 29 amino acids, PCV2c by 3 amino acids, PCV2d by 26 amino acids and PCV2e (“divergent PCV2a”) by 27 amino acids. Administration of a vaccine including the chimeric capsid polypeptide 3cl.14 remarkably results in cross protection against the most important currently pathogenic strains PCV2b and PCV2d. In certain embodiments, a vaccine is provided that includes one or more chimeric PCV2 capsid proteins and derivatives thereof wherein each chimeric protein and derivative thereof is unique and varies from any contributing parental strain by 3-37 amino acids. In other embodiments, a vaccine is provided that includes one or more chimeric PCV2 capsid proteins and derivatives thereof wherein each chimeric protein and derivative thereof is unique and varies vary from any parental chimeric capsid polypeptide by 1-27 amino acids. In certain embodiments, recombinant PCV2 capsid polypeptide and immunogenic derivatives thereof are provided that include capsid-derived amino acid sequences of at least PCV2c and PCV2d. In certain embodiments, recombinant PCV2 capsid polypeptides are provided that are expressed in bacterial, yeast, mammalian, or insect cells. In certain embodiments the immunogenic compositions including recombinant PCV2 capsid polypeptides are subunit vaccines while in other embodiments the recombinant PCV2 capsid polypeptides are presented in the context of inactivated whole virus vaccines.

In other embodiments, a vaccine is provided that includes one or more derivatives of chimeric PCV2 capsid proteins wherein the derivatives vary from any parental chimeric capsid polypeptides by conservative substitutions. In other embodiments, derivatives of chimeric capsid proteins are provided that vary from any parental chimeric capsid polypeptide by one to twenty seven amino acids, preferably one to fifteen, one to ten, one to five, or one or two amino acids. In still other embodiments, immunogenic PCV2 capsid proteins are provided including amino acid substitutions selected from strain specific variant amino acids such as are shown highlighted on FIG. 3 to provide a capsid polypeptide derivative that when used as a vaccine provides cross protection to two or more of PCV2a, PCV2b, PCV2c, PCV2d, PCV2e strains or genotypes and newly arising variants thereof.

TABLE 2
Amino Acid difference between parental capsid
polypeptides and chimeric derivatives

| | PCV2- | PCV2- | PCV2- | PCV2- |
|---|---|---|---|---|
| | 3cl.12_2 | 3cl.13 | 3cl.14 | 3cl.4_2 |
| Parental strains ↓ | SID 6 | SID 4 | SID 8 | SID 2 |
| PCV2a, | 25 | 27 | 29 | 20 |
| ACN AF264042 | | | | |
| SEQ ID NO: 10 | | | | |
| PCV2b, | 22 | 20 | 29 | 7 |
| ACN GU799576 | | | | |
| SEQ ID NO: 12 | | | | |
| PCV2c, | 16 | 8 | 3 | 21 |
| ACN EU148503 | | | | |
| SEQ ID NO: 16 | | | | |
| PCV2d-1 | 14 | 23 | 26 | 16 |
| ACN AY181947 | | | | |
| SEQ ID NO: 14 | | | | |
| PCV2e | 31 | 31 | 37 | 30 |
| CN EF524533 | | | | |
| SEQ ID NO: 18 | | | | |

TABLE 3
Amino acid differences between chimeric capsid polypeptides

| | PCV2- | PCV2- | PCV2- | PCV2- |
|---|---|---|---|---|
| Chimeric virus | 3cl.12_2 | 3cl.13 | 3cl.14 | 3cl.4_2 |
| capsids | SID 6 | SID 4 | SID 8 | SID 2 |
| PCV2-3cl.12_2 | 0 | 11 | 12 | 16 |
| SID 6 | | | | |
| PCV2-3cl.13 | 11 | 0 | 22 | 18 |
| SID 4 | | | | |
| PCV2-3cl.14 | 12 | 22 | 0 | 27 |
| SID 8 | | | | |
| PCV2-3cl.4_2 | 16 | 18 | 27 | 0 |
| SID 2 | | | | |

### Example 3

### Chimeric Vaccine Compositions

Whether as a live virus vaccine, a modified live virus vaccine, inactivated virus vaccine, attenuated vaccine, plasmid DNA vaccine or a subunit polypeptide vaccine, an immunologically effective amount is administered. The “immunogenically effective” amount is sufficient to stimulate an immune response, i.e., to stimulate the production of humoral antibodies and/or to stimulate a cell-mediated response. The vaccine composition may include suitable adjuvants as well as one or more wetting or dispersing agents. Such wetting agents include non-ionic surfactants such as polyoxyethylene/polyoxypropylene block copolymers, i.e. those marketed under the mark PLURONIC® and available from BASF Corp. (Mt. Olive, N.J.). Other useful nonionic surfactants include polyoxyethylene esters such as polyoxyethylene sorbitan monooleate (polysorbate 80), available under the trademark TWEEN 80®.

Examples of suitable adjuvants include, without limitation, immunostimulating bio-compatible oils such as vegetable-based oils (i.e. corn oil, sesame oil, olive oil, soybean oil, safflower oil, cotton seed oil, rape seed oil, sunflower oil, jojoba oil, coconut oil, peanut oil or mixtures thereof), squalane (shark liver oil) or other metabolizable oils as well as adjuvant mixtures including an SP oil (an oil emulsion comprising a polyoxyethylene-polyoxypropylene block copolymer, squalane, polyoxyethylene sorbitan monooleate and a buffered salt solution), EMULSIGEN (MPV Laboratories, Omaha, Nebr., USA), MONTANIDE (Seppic, Paris, France). Other adjuvants include oil-in-water adjuvants, polymer and water adjuvants, water-in-oil adjuvants, an aluminum hydroxide adjuvants, and vitamin E adjuvants.

One example of a suitable SP oil adjuvant includes 1 mg of a polyoxyethylene-polyoxypropylene block copolymer such as PLURONIC L121, 2 mg of Squalane, 0.16 mg of the polyoxyethylene sorbitan monooleate (polysorbate 80) such as TWEEN 80 and 0.05 ml of phosphate buffered saline (PB S).

Other components of the composition may include pharmaceutical excipients and buffers including without limitation HEPES Diluent, Eagle's Earle's MEM growth medium, HEPES acid, sodium hydrogen carbonate, hydrochloric acid, sodium hydroxide, such preservative compounds as formalin and thimerosal and water for injection.

### Example 4

### Chimeric Capsid Specific Monoclonal Antibodies and Mimotope Vaccines

In one embodiment, the recombinant chimeric porcine circoviruses provided herein are used to as antigens to generate antibodies specific to epitopes on the capsid proteins of one or more of chimeric viruses PCV2-3cl13, PCV2-3cl14, PCV2-3cl4_2, and PCV2-3cl12_2. Where the antibodies are monoclonal antibodies, the methodology used to obtain hybridomas and monoclonal antibodies may follow the conventional method of lymphocyte fusion and hybridoma culture originally described by Kohler & Milstein Nature 256 (1975) 495-497. Other methods for preparing monoclonal antibodies are also known including phage display such as described by Barbas C F, et al. “Assembly of combinatorial antibody libraries on phage surfaces: the gene III site” Proc. Natl. Acad. Sci. U.S.A. 88 (18) (1991) 7978-82 and subsequently modified. The derived antibodies are utilized for diagnostics as well as vaccine development.

In the context of vaccine development, the derived monoclonal antibodies are screened against peptide libraries such as phage display peptide libraries generated in accordance with known technology such as originally described by Smith G R. “Filamentous fusion phage: novel expression vectors that display cloned antigens on the virion surface” Science 228 (4705) (1985) 1315-7. In one embodiment, the antibodies specific to epitopes on the capsid proteins of one or more of chimeric viruses PCV2-3cl13, PCV2-3cl14, PCV2-3cl42, and PCV2-3cl12_2 are adsorbed on microtiter plates and incubated with the phage display peptide library. Phages binding to the antibodies are isolated through iterative rounds of selection. Promising clones are selected including using computational matching studies if desired. Isolated and proven mimotopes may be tested in immunization studies with the mimotopes to prove molecular mimicry by generation of antibodies recognizing the capsid proteins. Mixtures of capsid epitope mimotopes specific to different PCV subtypes are then used as peptide antigens together with adjuvants or as multiple mimotope linear constructs to generated protective immune responses to several PCV2 strains.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1-37. (canceled)

38. A vaccine composition comprising a recombinant porcine circovirus type 2 (PCV2) capsid polypeptide or an immunogenic derivative thereof, wherein the recombinant porcine circovirus type 2 (PCV2) capsid polypeptide is a chimeric amino acid sequence that differs from any naturally occurring PCV2 capsid polypeptide and includes capsid-derived amino acid sequences from multiple PCV2 genotypes.

39. The vaccine composition of claim 1, wherein the recombinant PCV2 capsid polypeptide is selected from the group consisting of capsid polypeptides designated as 3cl.14 (SEQ ID NO: 8), 3cl.13 (SEQ ID NO: 4), 3cl.4_2 (SEQ ID NO: 2), 3cl.12_2 (SEQ ID NO: 6).

40. The vaccine composition of claim 1, wherein the recombinant PCV2 capsid polypeptide or immunogenic derivative thereof includes capsid-derived amino acid sequences from two or more of PCV2a, PCV2b, PCV2c, PCV2d and PCV2e parental genotypes.

41. The vaccine composition of claim 3, wherein the recombinant PCV2 capsid polypeptide or immunogenic derivative thereof varies from any contributing parental genotype by 3-37 amino acids.

42. The vaccine composition of claim 3, wherein the immunogenic derivative varies from any parental chimeric capsid polypeptide by 1-27 amino acids.

43. The vaccine composition of claim 3, wherein the recombinant PCV2 capsid polypeptide or immunogenic derivative thereof comprises capsid-derived amino acid sequences of at least PCV2c and PCV2d.

44. The vaccine composition of claim 1, wherein the composition includes a chimeric PCV1-2 virus comprising a recombinant PCV1 that encodes the recombinant PCV2 capsid polypeptide in place of the capsid polypeptide of PCV1.

45. The vaccine composition of claim 1, wherein the recombinant PCV2 capsid polypeptide is expressed in bacterial, yeast, mammalian, or insect cells.

46. The vaccine composition of claim 1, wherein the composition is selected from a subunit vaccine, an inactivated whole virus vaccine, a live virus vaccine, a modified live virus vaccine, and an attenuated live virus vaccine.

47. The vaccine composition of claim 1, further including at least one adjuvant selected from the group consisting of: an oil-in-water adjuvant, an oil emulsion adjuvant, a polymer and water adjuvant, a water-in-oil adjuvant, an aluminum hydroxide adjuvant, a vitamin E adjuvant and combinations thereof.

48. The vaccine composition of claim 10, wherein the oil emulsion adjuvant comprises a polyoxyethylene-polyoxypropylene block copolymer, squalane, polyoxyethylene sorbitan monooleate and a buffered salt solution (SP-oil).

49. The vaccine composition of claim 1, wherein the composition further comprises at least one additional antigen protective against one or more of a bacterial microorganism, a viral microorganism, and a parasitic microorganism that can cause disease in pig.

50. The vaccine composition of claim 12, wherein the bacterial microorganism is selected from one or more of Actinobacilllus pleuropneumoniae, Bordetella bronchiseptica, Brachyspira hyodysenteriae, Brucellosis (B. suis), Campylobacter spp., Clostridium spp., Escherichia coli, Erysipelothrix rhusiopathiae, Haemophilus parasuis, Isospora suis, Lawsonia intracellularis, Leptospira spp., Listeria monocytogenes, Mycoplasma hyorhinis, Mycoplasma hyosynoviae, Mycoplasma flocculare, Mycoplasma hyopneumoniae, Pasteurella multocida, Streptococcum suis, Staphylococcus spp. including S. aureus and S. hyicus, Salmonella spp. including S. choleraesuis and S. enteritidis, methicillin-resistant Staphylococcus aureus (MRSA), Trichinella spiralis, Toxoplasma gondii and Yersinia enterocolitica.

51. The vaccine composition of claim 12, wherein the viral microorganism is selected from one or more of African swine fever virus, classical swine fever virus (CSF), foot and mouth disease virus (FMDV), Nipah virus, porcine cytomegalovirus, porcine epidemic diarrhea virus (PEDV), porcine enteroviruses, encephalomyocarditis virus, porcine reproductive and respiratory syndrome virus (PRRSV), porcine parvovirus (PPV), porcine respiratory coronavirus (PRVV), pseudorabies virus (PRV) a.k.a. suid herpesvirus type 1, rotavirus, swine influenza virus (SIV), torque teno virus (TTV), and transmissible gastroenteritis virus of swine (TGEV).

52. The vaccine composition of claim 12, wherein the parasitic microorganism is selected from one or more of Ascaris suum, Balatidium coli, Toxiplasma gondii, and Cryptosporidium parvum.

53. A method of protecting a pig against infection by multiple PCV2 genotypes comprising administering to the pig an immunologically effective amount of the vaccine composition of claim 1 by one or more routes selected from the group consisting of parenterally, intranasally, intradermally and transdermally.

54. A vaccine composition comprising a recombinant chimeric porcine circovirus comprising a recombinant porcine circovirus type 1 (PCV1) that encodes a porcine circovirus type 2 (PCV2) capsid polypeptide in place of the capsid protein of PCV1, the PCV2 capsid polypeptide comprising epitopes from capsid polypeptides of multiple PCV2 genotypes.

55. The vaccine composition of claim 17, wherein the PCV2 capsid polypeptide is selected from the group consisting of the capsid polypeptides designated as 3cl.14 (SEQ ID NO: 8), 3cl.13 (SEQ ID NO: 4), 3cl.4_2 (SEQ ID NO: 2), 3cl.12_2 (SEQ ID NO:6) and derivatives thereof.

56. The vaccine composition of claim 17, wherein the vaccine is selected from a subunit vaccine, an inactivated whole virus vaccine, a live virus vaccine, a modified live virus vaccine, and an attenuated live virus vaccine.

57. The vaccine composition of claim 17, wherein the recombinant chimeric porcine circovirus is designated as PCV1_3cl.14 and is encoded by SEQ ID NO: 37.


SEQUENCE LISTING (39 sequences)

SEQ ID NO: 1 - DNA, 705 bp, Porcine circovirus
atgacgtatccaaggaggcgttaccggagaagaagacaccgcccccgcagccatcttggccagatcctccgccgccgcccctggctcgtccacccccgccaccgttaccgctggagaaggaaaaatggcatcttcaacacccgcctatcccgcaccttcggatatactatcaagcgaaccacagtcagaacgccctcctgggcggtggacatgatgagattcaatattaatgactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccagcccttctcctaccactcccgctactttacccccaaacctgtcctagattccactattgattacttccaaccaaacaacaaaagaaaccagctgtggctgagactacaaactgctggaaatgtagaccatgtaggcctcggacacgcctttcaaaacagtacaaatgcccaggcctacaatgtccgtgtaaccatgtatgtacaattcagagaatttaatcttaaagaccccccacttaaccctaaatga

SEQ ID NO: 2 - protein, 234 aa, Porcine circovirus
MTYPRRRYRRRRHRPRSHLGQILRRRPWLVHPRHRYRWRRKNGIFNTRLSRTFGYTIKRTTVRTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGHAFQNSTNAQAYNVRVTMYVQFREFNLKDPPLNPK

SEQ ID NO: 3 - DNA, 705 bp, Porcine circovirus
atgacgtatccaaggaggcgttaccggagaagaagacaccgcccccgcagccatcttggccatatcctccgccgccgcccctggctcgtccacccccgccaccgctaccgttggagaaggaaaaatggaatcttcaatgcccgcctctcccgctcctttgtttataccgttaatgcctcacaggtctcaccaccctcttgggcggtggacatgatgagatttaatattaatgactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccaacccttctcctaccactcccgctactttacccccaaacctgtccttgattccactattgattacttccaaccaaataacaaaagaaatcagctgtggatgagactacaaactactggaaatgtagaccatgtaggcctcggacacgcctttcaaaacagtacaaatgcccaggcctacaatgtccgtgtaaccatgtatgtacaattcagagaatttaatcttaaagaccccccacttaaccctaaatga

SEQ ID NO: 4 - protein, 234 aa, Porcine circovirus
MTYPRRRYRRRRHRPRSHLGHILRRRPWLVHPRHRYRWRRKNGIFNARLSRSFVYTVNASQVSPPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWMRLQTTGNVDHVGLGHAFQNSTNAQAYNVRVTMYVQFREFNLKDPPLNPK

SEQ ID NO: 5 - DNA, 705 bp, Porcine circovirus
atgacgtatccaaggaggcgttaccggagaagaagacaccgcccccgcagccatcttggccatatcctccgccgccgcccctggctcgtccacccccgccaccgctaccgttggagaaggaaaaatggaatcttcaatgcccgcctctcccgctcctttgtttataccgttaatgcctcacaggtctcaccaccctcttgggcggtggacatgatgagatttaatattaaccaatttcttcccccaggagggggctcaaaccccctcactgtgccctttgaatactacagaataaggaagattaaggttgaattctggccctgctccccaatcacccagggtgacaggggagtgggctccactgctgttattctagatgataactttgtaacaaaggccacagccctaacctatgacccctatgtaaactactcctcccgccataccataccccagcccttctcctaccactcccgctatttcacccccaaacctgtccttgataggacaatcgattacttccaacccaataacaaaagaaatcaactctggctgagactacaaactactggaaatgtagaccatgtaggcctcggcactgcgttcgaaaacagtaaatacgaccaggactacaatatccgtataaccatgtatgtacaattcagagaatttaatcttaaagaccccccacttaaccctaaatga

SEQ ID NO: 6 - protein, 234 aa, Porcine circovirus
MTYPRRRYRRRRHRPRSHLGHILRRRPWLVHPRHRYRWRRKNGIFNARLSRSFVYTVNASQVSPPSWAVDMMRFNINQFLPPGGGSNPLTVPFEYYRIRKIKVEFWPCSPITQGDRGVGSTAVILDDNFVTKATALTYDPYVNYSSRHTIPQPFSYHSRYFTPKPVLDRTIDYFQPNNKRNQLWLRLQTTGNVDHVGLGTAFENSKYDQDYNIRITMYVQFREFNLKDPPLNPK

SEQ ID NO: 7 - DNA, 702 bp, Porcine circovirus
atgacgtatccaaggaggcgttaccggagaagaagacacggcccccgcagccatcttggccagatcctccgccgccgcccctggctcgtccacccccgccaccgctaccgttggagaaggaaaaatggaatcttcaatgcccgcctctcccgctcctttgtttataccgttaatgcctcacaggtctcaccaccctcttgggcggtggacatgatgagatttaatattaaccaatttcttcccccaggagggggctcaaaccccctcactgtgccctttgaatactacagaataagaaaggttaaagtggaattctttgcaagatcccccatcacccaaggtgacaggggagtgggctccactgctgttattctaaatgataactttgtaacaaaggccacagccctaacctatgacccctatgtaaactactcctcccgccataccataacccaacccttctcctaccactcccgctactttacccccaaacctgtccttgattccactattgattacttccaaccaaataacaaaagaaatcagctgtggatgagactacaaactactggaaatgtagaccatgtaggcctcggacacgcctttcaaaacagtacaaatgcccaggcctacaatgtccgtgtaaccatgtatgtacaattcagagaatttaatcttaaagaccccccacttaaaccctaa

SEQ ID NO: 8 - protein, 233 aa, Porcine circovirus
MTYPRRRYRRRRHGPRSHLGQILRRRPWLVHPRHRYRWRRKNGIFNARLSRSFVYTVNASQVSPPSWAVDMMRFNINQFLPPGGGSNPLTVPFEYYRIRKVKVEFFARSPITQGDRGVGSTAVILNDNFVTKATALTYDPYVNYSSRHTITQPFSYHSRYFTPKPVLDSTIDYFQPNNKRNQLWMRLQTTGNVDHVGLGHAFQNSTNAQAYNVRVTMYVQFREFNLKDPPLKP

SEQ ID NO: 9 - DNA, 702 bp, Porcine circovirus
atgacgtatccaaggaggcgttaccgcagaagaagacaccgcccccgcagccatcttggccagatcctccgccgccgcccctggctcgtccacccccgccaccgctaccgttggagaaggaaaaatggcatcttcaacacccgcctctcccgcaccttcggatatactgtcaaggctaccacagtcagaacgccctcctgggcggtggacatgatgagatttaatattgacgactttgttcccccgggaggggggaccaacaaaatctctataccctttgaatactacagaataagaaaggttaaggttgaattctggccctgctcccccatcacccagggtgataggggagtgggctccactgctgttattctagatgataactttgtaacaaaggccacagccctaacctatgacccatatgtaaactactcctcccgccatacaatcccccaacccttctcctaccactcccgttacttcacacccaaacctgttcttgactccaccattgattacttccaaccaaataacaaaaggaatcagctttggatgaggctacaaacctctagaaatgtggaccacgtaggcctcggcactgcgttcgaaaacagtatatacgaccaggactacaatatccgtgtaaccatgtatgtacaattcagagaatttaatcttaaagaccccccacttaaaccctaa

SEQ ID NO: 10 - protein, 233 aa, Porcine circovirus
MTYPRRRYRRRRHRPRSHLGQILRRRPWLVHPRHRYRWRRKNGIFNTRLSRTFGYTVKATTVRTPSWAVDMMRFNIDDFVPPGGGTNKISIPFEYYRIRKVKVEFWPCSPITQGDRGVGSTAVILDDNFVTKATALTYDPYVNYSSRHTIPQPFSYHSRYFTPKPVLDSTIDYFQPNNKRNQLWMRLQTSRNVDHVGLGTAFENSIYDQDYNIRVTMYVQFREFNLKDPPLKP

SEQ ID NO: 11 - DNA, 702 bp, Porcine circovirus
atgacgtatccaaggaggcgttaccggagaagaagacaccgcccccgcagccatcttggccagatcctccgccgccgcccctggctcgtccacccccgccaccgttaccgctggagaaggaaaaatggcatcttcaacacccgcctatcccgcaccttcggatatactatcaagcgaaccacagtcagaacgccctcctgggcggtggacatgatgagattcaatattaatgactttcttcccccaggagggggctcaaacccccgctctgtgccctttgaatactacagaataagaaaggttaaggttgaattctggccctgctccccgatcacccagggtgacaggggagtgggctccagtgctgttattctagatgataactttgtaacaaaggccacagccctcacctatgacccctatgtaaactactcctcccgccataccataacccagcccttctcctaccactcccgctactttacccccaaacctgtcctagattccactattgattacttccaaccaaacaacaaaagaaaccagctgtggctgagactacaaactgctggaaatgtagaccacgtaggcctcggcactgcgttcgaaaacagtatatacgaccaggaatacaatatccgtgtaaccatgtatgtacaattcagagaatttaatcttaaagaccccccacttaacccttaa

SEQ ID NO: 12 - protein, 233 aa, Porcine circovirus
MTYPRRRYRRRRHRPRSHLGQILRRRPWLVHPRHRYRWRRKNGIFNTRLSRTFGYTIKRTTVRTPSWAVDMMRFNINDFLPPGGGSNPRSVPFEYYRIRKVKVEFWPCSPITQGDRGVGSSAVILDDNFVTKATALTYDPYVNYSSRHTITQPFSYHSRYFTPKPVLDSTIDYFQPNNKRNQLWLRLQTAGNVDHVGLGTAFENSIYDQEYNIRVTMYVQFREFNLKDPPLNP

SEQ ID NO: 13 - DNA, 705 bp, Porcine circovirus
atgacgtatccaaggaggcgttaccgaagacgaagacaccgcccccgcagccatcttggccaaatcctccgccgccgcccctggctcgtccacccccgccaccattaccgctggagaaggaaaaatggcatcttcaacacccgcctctcccgcaccatcggttatactgtcaaggctaccacagtcagaacgccctcctgggcggtggacatgatgagatttaatattaatgattttcttcccccaggagggggctcaaaccccctcactgtgccctttgaatactacagaataaggaagattaaggttgaattctggccctgctccccaatcacccagggtgacaggggagtgggctccactgctgttattctagatgataactttgtaacaaaggccacagccctaacctatgacccctatgtaaactactcctcccgccataccataccccagcccttctcctaccactcccgctatttcacccccaaacctgtccttgataggacaatcgattacttccaacccaataacaaaagaaatcaactctggctgagactacaaactactggaaatgtagaccatgtaggcctcggcactgcgttcgaaaacagtaaatacgaccaggactacaatatccgtataaccatgtatgtacaattcagagaatttaatcttaaagaccccccacttaaccctaagtga

SEQ ID NO: 14 - protein, 234 aa, Porcine circovirus
MTYPRRRYRRRRHRPRSHLGQILRRRPWLVHPRHHYRWRRKNGIFNTRLSRTIGYTVKATTVRTPSWAVDMMRFNINDFLPPGGGSNPLTVPFEYYRIRKIKVEFWPCSPITQGDRGVGSTAVILDDNFVTKATALTYDPYVNYSSRHTIPQPFSYHSRYFTPKPVLDRTIDYFQPNNKRNQLWLRLQTTGNVDHVGLGTAFENSKYDQDYNIRITMYVQFREFNLKDPPLNPK

SEQ ID NO: 15 - DNA, 705 bp, Porcine circovirus
atgacgtatccaaggaggcgttaccggagaagaagacaccgcccccgcagccatcttggccatatcctccgccgccgcccctggctcgtccacccccgccaccgctaccgttggagaaggaaaaatggaatcttcaatgcccgcctctcccgctcctttgtttataccgttaatgcctcacaggtctcaccaccctcttgggcggtggacatgatgagatttaatattaaccaatttcttcccccaggagggggctcaaaccccctcactgtgccctttgaatactacagaataagaaaggttaaagtggaattctttgcaagatcccccatcacccaaggtgacaggggagtgggctccactgctgttattctaaatgataactttgtaacaaaggccacagccctaacctatgacccctatgtaaactactcctcccgccataccataacccaacccttctcctaccactcccgctactttacccccaaacctgtccttgattccactattgattacttccaaccaaataacaaaagaaatcagctgtggatgagactacaaactactggaaatgtagaccatgtaggcctcggacacgcctttcaaaacagtacaaatgcccaggcctacaatgtccgtgtaaccatgtatgtacaattcagagaatttaatcttaaagaccccccacttaaccctaagtga

SEQ ID NO: 16 - protein, 234 aa, Porcine circovirus
MTYPRRRYRRRRHRPRSHLGHILRRRPWLVHPRHRYRWRRKNGIFNARLSRSFVYTVNASQVSPPSWAVDMMRFNINQFLPPGGGSNPLTVPFEYYRIRKVKVEFFARSPITQGDRGVGSTAVILNDNFVTKATALTYDPYVNYSSRHTITQPFSYHSRYFTPKPVLDSTIDYFQPNNKRNQLWMRLQTTGNVDHVGLGHAFQNSTNAQAYNVRVTMYVQFREFNLKDPPLNPK

SEQ ID NO: 17 - DNA, 702 bp, Porcine circovirus
atgacgtatccaaggaggcgttaccggagaagaagacacggcccccgcagccatcttggccatatcctccgccgccgcccctggctcgtccacccccgccaccgttaccgctggagaaggaaaaatggcattttcaacagccgcctctcccgcaccttcggatatactgtcaaggctaccacagtcacaacgccctcctgggcggtggacatgctgagattcaatattgacgactttcttcccccgggaggggggaccaacaaaatctctataccccttgaatactacagaataagaaaggttaaggttgaattctggccctgctccccaatcacccagggtgacaggggagttggatccagtgctgtaattctagatgataactttttccctaagtccacagccctaacctatgacccctacgtaaactactcctcccgccataccataccccagcccttctcctaccactcccgctacttcacccccaaacctgtccttgattccaccattgattacttccaaccaaataacaaaaggaatcagctgtggatgagaattcaaaccagtaaaaatgtagaccacgtaggcctcggcactgcgttcgaaaacagtaaatacgaccaggactacaatatccgtgtaaccatgtatgtacaattcagagaatttaatcttaaagaccccccacttaaaccctaa

SEQ ID NO: 18 - protein, 233 aa, Porcine circovirus
MTYPRRRYRRRRHGPRSHLGHILRRRPWLVHPRHRYRWRRKNGIFNSRLSRTFGYTVKATTVTTPSWAVDMLRFNIDDFLPPGGGTNKISIPLEYYRIRKVKVEFWPCSPITQGDRGVGSSAVILDDNFFPKSTALTYDPYVNYSSRHTIPQPFSYHSRYFTPKPVLDSTIDYFQPNNKRNQLWMRIQTSKNVDHVGLGTAFENSKYDQDYNIRVTMYVQFREFNLKDPPLKP

SEQ ID NO: 19 - DNA, 31 bp, Porcine circovirus
ttactgagtcttttttatcacttcgtaatgg

SEQ ID NO: 20 - DNA, 35 bp, Porcine circovirus
ctttcgttttcagatatgacgtatccaaggaggcg

SEQ ID NO: 21 - DNA, 30 bp, Porcine circovirus
acccattacgaagtgataaaaaagactcag

SEQ ID NO: 22 - DNA, 29 bp, Porcine circovirus
agcccgcggaaatttctgacaaacgttac

SEQ ID NO: 23 - DNA, 29 bp, Porcine circovirus
tttccgcgggctggctgaacttttgaaag

SEQ ID NO: 24 - DNA, 33 bp, Porcine circovirus
cttttttgttatcacatcgtaatggtttttatt

SEQ ID NO: 25 - DNA, 34 bp, Porcine circovirus
ttctttcacttttataggatgacgtatccaagga

SEQ ID NO: 26 - DNA, 36 bp, Porcine circovirus
cctccttggatacgtcatcctataaaactgaaagaa

SEQ ID NO: 27 - DNA, 34 bp, Porcine circovirus
aaataaaaaccattacgatgtgataacaaaaaag

SEQ ID NO: 28 - DNA, 20 bp, Porcine circovirus
gaggtgttcggccctcctca

SEQ ID NO: 29 - DNA, 20 bp, Porcine circovirus
aaaagcaaatgggctgctaa

SEQ ID NO: 30 - DNA, 20 bp, Porcine circovirus
tggtaaccatcccaccactt

SEQ ID NO: 31 - DNA, 18 bp, Porcine circovirus
tggagaagaagttgttgt

SEQ ID NO: 32 - DNA, 19 bp, Porcine circovirus
tctacagtcaatggatacc

SEQ ID NO: 33 - DNA, 1767 bp, Porcine circovirus
cttttttatcacttcgtaatggtttttattattcatttagggttaagtggggggtctttaagattaaattctctgaattgtacatacatggttacacggacattgtaggcctgggcatttgtactgttttgaaaggcgtgtccgaggcctacatggtctacatttccagcagtttgtagtctcagccacagctggtttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattgaatctcatcatgtccaccgcccaggagggcgttctgactgtggttcgcttgatagtatatccgaaggtgcgggataggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccatatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattactttcttggtattttggaagaatgctacagaacaatccacggaggaagggggccagttcgtcaccctttcccccccatgccctgaatttccatatgaaataaattactgagt

SEQ ID NO: 34 - DNA, 1767 bp, Porcine circovirus
cttttttatcacttcgtaatggtttttattattcatttagggttaagtggggggtctttaagattaaattctctgaattgtacatacatggttacacggacattgtaggcctgggcatttgtactgttttgaaaggcgtgtccgaggcctacatggtctacatttccagtagtttgtagtctcatccacagctgatttcttttgttatttggttggaagtaatcaatagtggaatcaaggacaggtttgggggtaaagtagcgggagtggtaggagaagggttgggttatggtatggcgggaggagtagtttacataggggtcataggtgagggctgtggcctttgttacaaagttatcatctagaataacagcactggagcccactcccctgtcaccctgggtgatcggggagcagggccagaattcaaccttaacctttcttattctgtagtattcaaagggcacagagcgggggtttgagccccctcctgggggaagaaagtcattaatattaaatctcatcatgtccaccgcccaagagggtggtgagacctgtgaggcattaacggtataaacaaaggagcgggagaggcgggcattgaagattccatttttccttctccaacggtagcggtggcgggggtggacgagccaggggcggcggcggaggatatggccaagatggctgcgggggcggtgtcttcttctccggtaacgcctccttggatacgtcatatctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccatatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattactttcttggtattttggaagaatgctacagaacaatccacggaggaagggggccagttcgtcaccctttcccccccatgccctgaatttccatatgaaataaattactgagt

SEQ ID NO: 35 - DNA, 1767 bp, Porcine circovirus
cttttttatcacttcgtaatggtttttattattcatttagggttaagtggggggtctttaagattaaattctctgaattgtacatacatggttatacggatattgtagtcctggtcgtatttactgttttcgaacgcagtgccgaggcctacatggtctacatttccagtagtttgtagtctcagccagagttgatttcttttgttattgggttggaagtaatcgattgtcctatcaaggacaggtttgggggtgaaatagcgggagtggtaggagaagggctggggtatggtatggcgggaggagtagtttacataggggtcataggttagggctgtggcctttgttacaaagttatcatctagaataacagcagtggagcccactcccctgtcaccctgggtgattggggagcagggccagaattcaaccttaatcttccttattctgtagtattcaaagggcacagtgagggggtttgagccccctcctgggggaagaaattggttaatattaaatctcatcatgtccaccgcccaagagggtggtgagacctgtgaggcattaacggtataaacaaaggagcgggagaggcgggcattgaagattccatttttccttctccaacggtagcggtggcgggggtggacgagccaggggcggcggcggaggatatggccaagatggctgcgggggcggtgtcttcttctccggtaacgcctccttggatacgtcatatctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccatatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattactttcttggtattttggaagaatgctacagaacaatccacggaggaagggggccagttcgtcaccctttcccccccatgccctgaatttccatatgaaataaattactgagt

SEQ ID NO: 36 - DNA, 1768 bp, Porcine circovirus
cttttttatcacttcgtaatggtttttattattcatttagggtttaagtggggggtctttaagattaaattctctgaattgtacatacatggttacacggacattgtaggcctgggcatttgtactgttttgaaaggcgtgtccgaggcctacatggtctacatttccagtagtttgtagtctcatccacagctgatttcttttgttatttggttggaagtaatcaatagtggaatcaaggacaggtttgggggtaaagtagcgggagtggtaggagaagggttgggttatggtatggcgggaggagtagtttacataggggtcataggttagggctgtggcctttgttacaaagttatcatttagaataacagcagtggagcccactcccctgtcaccttgggtgatgggggatcttgcaaagaattccactttaacctttcttattctgtagtattcaaagggcacagtgagggggtttgagccccctcctgggggaagaaattggttaatattaaatctcatcatgtccaccgcccaagagggtggtgagacctgtgaggcattaacggtataaacaaaggagcgggagaggcgggcattgaagattccatttttccttctccaacggtagcggtggcgggggtggacgagccaggggcggcggcggaggatctggccaagatggctgcgggggccgtgtcttcttctccggtaacgcctccttggatacgtcatatctgaaaacgaaagaagtgcgctgtaagtattaccagcgcacttcggcagcggcagcacctcggcagcacctcagcagcaacatgcccagcaagaagaatggaagaagcggaccccaaccacataaaaggtgggtgttcacgctgaataatccttccgaagacgagcgcaagaaaatacgggagctcccaatctccctatttgattattttattgttggcgaggagggtaatgaggaaggacgaacacctcacctccaggggttcgctaattttgtgaagaagcaaacttttaataaagtgaagtggtatttgggtgcccgctgccatatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattactttcttggtattttggaagaatgctacagaacaatccacggaggaagggggccagttcgtcaccctttcccccccatgccctgaatttccatatgaaataaattactgagt

SEQ ID NO: 37 - DNA, 1764 bp, Porcine circovirus
cttttttgttatcacatcgtaatggtttttattattcatttagggtttaagtggggggtctttaagattaaattctctgaattgtacatacatggttacacggacattgtaggcctgggcatttgtactgttttgaaaggcgtgtccgaggcctacatggtctacatttccagtagtttgtagtctcatccacagctgatttcttttgttatttggttggaagtaatcaatagtggaatcaaggacaggtttgggggtaaagtagcgggagtggtaggagaagggttgggttatggtatggcgggaggagtagtttacataggggtcataggttagggctgtggcctttgttacaaagttatcatttagaataacagcagtggagcccactcccctgtcaccttgggtgatgggggatcttgcaaagaattccactttaacctttcttattctgtagtattcaaagggcacagtgagggggtttgagccccctcctgggggaagaaattggttaatattaaatctcatcatgtccaccgcccaagagggtggtgagacctgtgaggcattaacggtataaacaaaggagcgggagaggcgggcattgaagattccatttttccttctccaacggtagcggtggcgggggtggacgagccaggggcggcggcggaggatctggccaagatggctgcgggggccgtgtcttcttctccggtaacgcctccttggatacgtcatcctataaaagtgaaagaagtgcgctgctgtagtattaccagcgcacttcggcagcggcagcacctcggcagcgtcagtgaaaatgccaagcaagaaaagcggcccgcaaccccataagaggtgggtgttcacccttaataatccctccgaggaggagaaaaacaaaatacgggagcttccaatctccctttttgattattttgtttgcggagaggaaggtttggaagagggtagaactcctcacctccaggggtttgcgaattttgctaagaagcagacttttaacaaggtgaagtggtattttggtgcccgctgccacatcgagaaagcgaaaggaaccgaccagcagaataaagaatactgcagtaaagaaggccacatacttatcgagtgtggagctccgcggaaccaggggaagcgcagcgacctgtctactgctgtgagtacccttttggagacggggtctttggtgactgtagccgagcagttccctgtaacgtatgtgagaaatttccgcgggctggctgaacttttgaaagtgagcgggaagatgcagcagcgtgattggaagacagctgtacacgtcatagtgggcccgcccggttgtgggaagagccagtgggcccgtaattttgctgagcctagcgacacctactggaagcctagtagaaataagtggtgggatggatatcatggagaagaagttgttgttttggatgatttttatggctggttaccttgggatgatctactgagactgtgtgaccggtatccattgactgtagagactaaagggggtactgttccttttttggcccgcagtattttgattaccagcaatcaggccccccaggaatggtactcctcaactgctgtcccagctgtagaagctctctatcggaggattactactttgcaattttggaagactgctggagaacaatccacggaggtacccgaaggccgatttgaagcagtggacccaccctgtgcccttttcccatataaaataaattactgagt

SEQ ID NO: 38 - DNA, 16 bp, synthetic
gtaaaacgacggccag

SEQ ID NO: 39 - DNA, 17 bp, synthetic
caggaaacagctatgac